(19) United States
(12) Patent Application Publication
(10) Pub. No.: US 2018/0050097 A1
(43) Pub. Date: Feb. 22, 2018

(54) IMMUNOTHERAPY COMPOSITIONS AND METHODS FOR TREATMENT OF TAUOPATHY AND TRANSGENIC MOUSE

(71) Applicant: STC. UNM, Albuquerque, NM (US)

(72) Inventors: Kiran Bhaskar, Albuquerque, NM (US); Nicole Maphis, Albuquerque, NM (US); David S. Peabody, Albuquerque, NM (US); Bryce Chackerian, Albuquerque, NM (US); Julianne Peabody, Albuquerque, NM (US); Erin Crossey, Albuquerque, NM (US)

(21) Appl. No.: 15/561,130

(22) PCT Filed: Mar. 25, 2016
(86) PCT No.: PCT/US2016/024174

§ 371 Date: Sep. 25, 2017

Related U.S. Application Data
(60) Provisional application No. 62/138,015, filed on Mar. 25, 2015.
(60) Provisional application No. 62/153,099, filed on Apr. 27, 2015.

(51) Int. Cl. A61K 39/00; A01K 67/027; C12N 7/00

(52) U.S. Cl. CPC A61K 39/0007; A01K 67/0278; C12N 7/00; A61K 2039/5258; A61K 2039/575; C12N 2795/18123; A01K 2267/0312; C12N 2795/18134; A01K 2217/056; A01K 2217/15; A01K 2217/075; A01K 2267/0368; A01K 2267/0306; C12N 2795/18171

(57) ABSTRACT

This disclosure describes, in one aspect, immunogens effective for treating and/or diagnosing tauopathy, and immunotherapeutic compositions and methods involving those immunogens. Generally, the immunogen includes an antigen presentation component and a microtubule-associated tau protein (MAPT) component linked to at least a portion of the antigen presentation component. This disclosure describes, in another aspect, a transgenic mouse. Generally, the transgenic mouse possesses brain cells that have a polynucleotide that encodes human microtubule-associated protein tau (MAPT). The polynucleotide further exhibits a deletion of at least a portion of endogenous mouse MAPT. The transgenic mouse also includes a forebrain neuron-specific deletion of a polynucleotide that encodes Myeloid Differentiation Primary Response Gene 88 (MyD88). In a further aspect, this disclosure describes a method of producing the transgenic mouse.

### CROSS-REFERENCE TO RELATED APPLICATIONS

This application claims priority to U.S. Provisional Patent Application Ser. No. 62/138,015, filed Mar. 25, 2015 and U.S. Provisional Patent Application Ser. No. 62/153,099, filed Apr. 27, 2015, each of which is incorporated herein by reference.

### GOVERNMENT FUNDING

This invention was made with government support under NS077089, awarded by the National Institutes of Health. The government has certain rights in the invention.

### SEQUENCE LISTING

This application contains a Sequence Listing electronically submitted to the United States Patent and Trademark Office via EFS-Web as an ASCII text file entitled “310-0990201 ST25.txt” having a size of 20 kilobytes and created on Mar. 23, 2016. Due to the electronic filing of the Sequence Listing, the electronically submitted Sequence Listing serves as both the paper copy required by 37 CFR §1.821(c) and the CRF required by §1.821(e). The information contained in the Sequence Listing is incorporated by reference herein.

### SUMMARY

This disclosure describes, in one aspect, an immunogen effective for treating or diagnosing tauopathy. Generally, the immunogen includes an antigen presentation component and a microtubule-associated tau protein (MAPT) component linked to at least a portion of the antigen presentation component.

In some embodiments, the MAPT component can include at least one amino acid residue modified to include a PO₃H₂ group.

In some embodiments, the antigen presentation component can include a virus-like particle (VLP). In some embodiments, virus-like particle (VLP) can include bacteriophage Qβ or MS2.

In some embodiments, the antigen presentation component may be covalently linked to the MAPT component. In some embodiments, the covalent link can include a succinimidyl-6-[β-maleimidopropionamido]hexanoate (SMPH) linkage.

In another aspect, this disclosure describes pharmaceutical compositions that include an immunogen as described herein. The pharmaceutical composition can further include an adjuvant.

In another aspect, this disclosure describes a method of treating a subject having or at risk of having a tauopathic condition. Generally, the method includes administering to the subject an amount of an immunogen as described herein effective to ameliorate at least one symptom or clinical sign of the tauopathic condition.

In some embodiments, the tauopathic condition can include Alzheimer's disease, progressive supranuclear palsy (PSP), corticobasal degeneration (CBD), Pick's disease (PiD), frontotemporal dementia and Parkinsonism linked to chromosome-17 Tau Type (FTDP-17T), argyrophilic grain dementia (AGD), traumatic brain injury (TBI), or chronic traumatic encephalopathy (CTE).

In some embodiments, the symptom or clinical sign of the tauopathic condition can include neurodegeneration or cognitive impairment.

In some embodiments, the method can further include at least one anti-inflammatory strategy such as, for example, enrichment of IgG4 immunoglobulins, removing RNA from the VLP component, or enrichment of regulatory B cells that express IL-10.

In some embodiments, the treatment can be prophylactic. In other embodiments, the treatment can be therapeutic.

In another aspect, this disclosure describes a polynucleotide that encodes an immunogen as described herein.

In another aspect, this disclosure describes a cell that includes the polynucleotide summarized immediately above.

In a further aspect, this disclosure describes a transgenic mouse. Generally, the transgenic mouse possesses brain cells that have a polynucleotide that encodes human microtubule-associated protein tau (MAPT). The polynucleotide further exhibits a deletion of at least a portion of endogenous mouse MAPT. The transgenic mouse also includes a forebrain neuron-specific deletion of a polynucleotide that encodes Myeloid Differentiation Primary Response Gene 88 (MyD88).

In another aspect, this disclosure describes a method of producing the transgenic mouse.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Chemical conjugation of peptides to Qβ virus-like particles (VLPs). (A) Schematic representation of the conjugation protocol. The bifunctional cross-linker (SMPH; Pierce, Thermo Fisher Scientific, Inc., Waltham, Mass.) reacts with primary amine groups on the surface of Qβ VLPs (indicated in (B)). After purification, the phage is reacted with target peptides containing cysteine. (C) List of pathological microtubule-associated tau proteins (MAPTs) conjugated to Qβ VLPs. (D) Conjugation extent is determined by analysis of denatured particles by gel electrophoresis. Qβ coat protein modified by conjugation of one or more peptides displays a mobility shift on a 10% Nu-Page gel. A short MAPT peptide (13mer; phosphorylated at T181, S199/S202, and S396/S404) has been successfully attached to Qβ VLP. Individual coat protein subunits are modified with one or two peptides. Anti-MAPT^{pT181}, anti-MAPT^{pS199/S202}, and anti-MAPT^{pS396/S404} antibody levels in C57Bl/6 mice immunized intramuscularly with two doses of 10 μg of MAPT peptide conjugated Qβ VLP. Each data point is an individual mouse. Lines show geometric mean titers for each group. All three conjugated VLPs induce high titer IgG against respective MAPT peptides. Control mice immunized with Qβ VLPs alone had no detectable IgG titer.

FIG. 2. Immunization of 2-month-old rTg4510 mice with Qβ-MAPT^{pT181} showed reduced MAPT pathology and improved memory as well as attenuated microglial inflammation versus rTg4510 mice immunized with Qβ alone. (A and C): Brain sections (30 μm) from rTg4510 mice vaccinated with either Qβ (control) or Qβ-MAPT^{pT181} show reduced phospho-MAPT phosphorylated at T231 (recognized by AT180 antibody) in Qβ-MAPT^{pT181} versus control. (B-E): A series of low-power images of brain sections stained with Gallyas silver (to detect neurofibrillary tangles (NFTs)) were used to reconstruct the cortex and hippocampus. Four sections/animal were used for quantification in E. Statistically (*p<0.05) significant reduction in the Gallyas silver positive neurons in the cortex of Qβ-MAPT^{pT181} mice compared to controls was observed. (F) Vaccinated rTg4510 mice also show improved spatial memory (indicated by mice staying in the target quadrant on a test day after 5 days of learning in the Morris Water Maze test; **p<0.01) compared to controls. (G) Reduced microglial inflammation, as evidenced by microglial activation marker CD45, is observed in the Qβ-MAPT^{pT181} mice compared to non-vaccinated or control groups.

FIG. 3. (A-D) Blast exposure characteristics for five animals; (E) Photographic images of Single air blast injury shock tube device.

FIG. 4. Single air blast injury (SAI) induces robust AT8-site MAPT hyperphosphorylation in the hippocampus (A and top panel in B) and temporal cortex (bottom panel B) 48 h post injury. Scale bar 250 μm in A; 100 μm in B (top panel); 10 μm in B (bottom panel). CX=Temporal cortex.

FIG. 5. Injury cavity, T2 MRI, microglial activation and tau pathology following single moderate to severe TBI. Two-month-old non-transgenic (WT) or hTau mice were exposed to lateral fluid percussion injury (FPI) and sacrificed 3 days post injury (DPI). Note the presence of injury cavity in both WT and hTau mice. In a separate study, closed contusion injury (CCI) was administered to B6 mice and T2 weighed Mill analysis was performed 42 DPI.

FIG. 6. Schematic diagram illustrating conjugation of MAPT peptides to VLPs.

FIG. 7. A schematic diagram with timeline and group size for Single Air-blast Injury (SAI) studies.

FIG. 8. Schematic depicting a table with different groups, their genotypes, group size, rationale for selecting them as well as immunotherapy experimental design for inoculation of non-transgenic and hTau mice with different MAPT-VLPs.

FIG. 9. Cxc3cr1 deficiency induces MAPT hyperphosphorylation, aggregation, microglial activation as well as behavioral impairments in hTau mice. (A) Western blot analysis of hippocampal lysates from 6-month-old hTauCx3cr1^{+/+} and hTauCx3cr1^{-/-} mice revealed an increase in MAPT phosphorylation at AT8, AT180 and PHF1 sites in hTauCx3cr1^{-/-} mice, but no alterations in total MAPT levels. (B-G) Numerous AT8 immunoreactive CA3 neurons (D) also displayed Gallyas silver positive MAPT aggregates (G) in hTauCx3cr1^{-/-} mice compared to age-matched non-transgenic controls (Non-Tg) (B, E) or hTauCx3cr1^{+/+} (C, F) mice. Altered morphology of Iba1+ microglia in the hippocampus of hTauCx3cr1^{-/-} mice (J) compared to Non-Tg (H) or hTau-Cx3cr1^{+/+} mice (I). (K) Levels of Sarkosyl insoluble and AT8 positive MAPT are higher in the hippocampus of hTauCx3cr1^{-/-} mice compared to hTauCx3cr1^{+/+} mice. (L) Y maze testing revealed a significant deficit in spontaneous alternation (working memory) in the hTauCx3cr1^{-/-} mice at six months of age when compared to either Cx3cr1^{-/-} or hTauCx3cr1^{+/+} mice.

FIG. 10. Schematic of one embodiment of a vaccination strategy.

FIG. 11. Antibody titers for mice immunized according to the protocol of FIG. 10 with Qβ alone; Qβ-MAPT^{pT181}; or MAPT^{pT181} with alum adjuvant (AA) (without the Qβ VLP platform).

FIG. 12. pTau antibodies are detected in the brain. (A) pTau-VLP (Qβ-MAPT^{pT181}) vaccine induced a 5-fold induction of anti pT181 IGg titers in the serum compared to VLP (%) alone in rTg4510 mice: a very strong antibody response. The result was similar in non-transgenic mice. (B) Higher levels of anti-pT181 IgG antibodies (measured by ELISA) were found in cortical lysates of non-transgenic mice vaccinated with Qβ-MAPT^{pT181} than in cortical lysates of non-transgenic mice vaccinated with VLP (Qβ) alone. (C) The presence of anti-pT181 antibodies in the brain parenchyma of vaccinated non-Tg mice was visualized by reverse immunohistochemistry.

FIG. 13. Vaccination improves novel object recognition. The novel object recognition test was performed as described in Example 3. Wild type mice typically spend about 80% of their time with the novel object. In contrast, untreated rTg4510 mice typically spend relatively equal amounts of time with both the novel object and the familiar object (A) Qβ-MAPT^{pT181}-vaccinated rTg4510 mice show a statistically significant increased preference to the novel object (compared to a familiar object). (B) Qβ-MAPT^{pS396/S404}-vaccinated mice show a statistically significant increased preference to the novel object (compared to a familiar object). (C) Qβ-MAPT^{pS199/pS202} vaccinated rTg4520 mice show a statistically significant increased preference to the novel object (compared to a familiar object). In each case, rTg4520 mice vaccinated with Qβ alone showed no significant preference to the novel object.

FIG. 14. Vaccination decreases p-tau. (A) Western blot analysis of proteins extracted from mice brains using antibodies specific to phosphorylated MAPT pS199/S202 (AT8) showed that rTg4510 mice injected with Qβ-MAPT^{pT181} displayed significantly reduced levels of AT8+ MAPT compared to Qβ-injected rTg4510 mice. (B) Quantification of the Western Blot results showed Qβ-MAPT^{pT181} vaccination significantly decreased hyperphosphorylated tau, by 3-fold. (C) Immunohistochemistry using antibodies specific to phosphorylated MAPT pS199/S202 (AT8) of the CA3 region of the hippocampus showed reduced phosphorylated tau in animals vaccinated with pTau-VLP (Qβ-MAPT^{pT181}) compared to animals vaccinated with VLP (Qβ).

FIG. 15. Non-Tg mice have larger cortices than rTg4510 mice, but treatment with Qβ-MAPT^{pT181} reduces cortical atrophy in rTg4510 mice. (A) MRI analysis (4.7T) of the cortex of control (non-Tg) and vaccinated (VLP and pTau-VLP) mice was performed. Cortical volume was approximated by sequentially measuring sections throughout the cortex and multiplying the area by 0.5 mm (slice thickness). (B, C) At 6 months of age (2 months post therapy), a trend towards reduced brain atrophy was observed in rTg4510 mice vaccinated with Qβ-MAPT^{pT181} compared to rTg4510 mice vaccinated with Qβ.

FIG. 16. (Upper panels) Qβ-MAPT^{pT181} (pTau-VLP) vaccinated rTg4510 mice display a lesser degree of age-related brain atrophy than Qβ (VLP) vaccinated rTg4510mice, based on statistical strength. (Lower panels) Two group comparative analysis via unpaired student t-test shows statistically significant rescue in cortical atrophy in 4 month old rTg4510 mice treated with Qβ-MAPT^{pT181} compared to 4 month old Qβ treated control mice.

FIG. 17. Neurons in the hippocampus of rTg4510 mice immunized with Qβ-MAPT^{pT181} show a marked reduction in Gallyas silver positive tangles compared to rTg4510 mice immunized with Qβ alone. Quantification is shown in the right panel (n=3 for Qβ (VLP); n=5 for Qβ-MAPT^{pT181} (pTau-VLP)).

FIG. 18. Vaccination decreases Sarkosyl-insoluble neurofibrillary tangles (NFTs). Significantly reduced proportions of Sarkosyl-insoluble tangles to Sarkosyl-soluble tangles were observed in the Qβ-MAPTpT181-vaccinated mice compared to Qβ-treated control mice (A) Western blot of Sarkosyl insoluble AT8 (phosphorylated tau) and Tau12 (human tau) in VLP (Qβ vaccinated) mice and p-Tau-VLP (Qβ-MAPTpT181 vaccinated) mice. (B) Western blot of Sarkosyl soluble AT8 and Tau12 in VLP mice and p-Tau-VLP mice. (C) Ratios of insoluble to soluble AT8 and Tau12 in VLP mice and p-Tau-VLP mice.

FIG. 19. Vaccination reduces neuroinflammation and neuronal apoptosis. Upper panel: a strong decrease in CD45+ immunoreactivity, indicating decreased neuroinflammation, was observed in the hippocampus of rTg4510 mice vaccinated with Qβ-MAPT^{pT181} versus the hippocampus of rTg4510 mice vaccinated with Qβ. Lower panel: Immunofluorescence analysis of NeuN (marker of neuron) and TUNEL (marker for apoptotic cells) in the hippocampus was used to determine if Qβ-MAPT^{pT181} vaccination reduces neurodegeneration/neuronal loss. rTg4510 mice vaccinated with Qβ-MAPT^{pT181} showed reduced numbers of TUNEL-NeuN double-positive cells versus rTg4510 mice vaccinated with Qβ.

FIG. 20. Vaccinated mice show no evidence of brain hemorrhage. The brain sections from spontaneously-hypertensive stroke prone rats (SHR-SP rats), and rTg4510 mice vaccinated with Qβ-MAPT^{pT181} or control Qβ were stained with Haematoxylin and Eosin (H&E). The brains of SHR-SP rats display a significant level of brain hemorrhage (indicated with arrows). No evidence of hemorrhage in the brains of mice vaccinated with Qβ-MAPT^{pT181} or Qβ was observed.

FIG. 21. Vaccination with Qβ-MAPTP^{S396/S404} reduces phosphorylation of tau. (A) Western blot analysis of hippocampal lysates from rTg4510 mice vaccinated with Qβ-MAPT^{pS396/S404} and Qβ alone show modest reduction in AT8+ and AT180+ tau in mice immunized with Qβ-MAPT^{pS396/S404} compared to Qβ-treated controls; results are quantified in (B).

FIG. 22. Working model shows extracellular tau acting as a primary driver of inflammation (similar to other known inflammasome activators—ATP, K+ efflux, and mitochondrial-reactive oxygen species (ROS), which are released in response to cell death) in IL-1β-mediated neuroinflammation and neurotoxicity.

FIG. 23. A schematic diagram of TLR4 signaling, including through MyD88.

FIG. 24. AT180 and PHF1 positive p-Tau and 98 kDa oligomeric form of tau levels are significantly reduced in hTauCamK2αCreMyD88^{f/f} mice compared to age-matched hTau mice.

FIG. 25. (A) Schematic showing the novel object recognition (NOR) test in mice. NOR is a 3-day test to measure ‘recognition memory. During the first two days, mice are allowed to explore two identical objects. On the third day (test day), one object is replaced with a novel object and the time spent with each object is scored. (B) Six month old non-transgenic (Non-Tg), MyD88^{f/f} and hTauCamK2αCreMyD88^{f/f} mice spend significantly more time with novel object compared to hTau mice.

FIG. 26. A schematic diagram of the mating scheme used to produce the hTauCamK2αCreMyD88^{f/f} mouse.

FIG. 27 shows the effect of immunization of hTau″ mice with Qβ or Qβ-MAPT^{pT181} on anti-pTau IgG titer. Statistically significant increased titers for anti-pTau IgG were observed in the sera of 6 month old hTau mice after immunization with MAPT^{pT181} compared to mice vaccinated with Qβ alone.

FIG. 28 (A) A trend towards reduced positive correlation between apparent diffusion coefficient (ADC) and fractional anisotropy (FA) in Qβ-MAPT^{pT181} vaccinated mice compared to Qβ vaccinated mice suggests protection from white matter damage in Qβ-MAPT^{pT181} vaccinated mice. (B) Correlation of apparent diffusion coefficient (ADC) and fractional anisotropy (FA) in the corpus collosum of non-transgenic mice (circles), Qβ-MAPT^{pT181} vaccinated rTg4510 mice (squares) and Qβ vaccinated rTg4510 mice (diamonds).

FIG. 29. (A, B, D, E) Fluid percussion injury (FPI) resulted in activation of Iba1+ microglia and hyperphosphorylation of MAPT at AT8 site in both 2-month-old WT and hTau mice 3 days post-injury (3 DPI). Notably, administration of FPI to hTau mice resulted in more pronounced AT8 reactivity in neurons. While mouse MAPT in WT mice did not show any silver-positive MAPT aggregates in response to traumatic brain injury (TBI) (C), silver-positive MAPT aggregates were observed in the brains of hTau mice following FPI (F). Scale bar 20 μm (in A, B, D, E) and 15 μm (in C and F).

### DETAILED DESCRIPTION OF ILLUSTRATIVE EMBODIMENTS

This disclosure describes compositions and methods for prophylactic and/or therapeutic treatment of tauopathy. This disclosure further describes a new mouse model that can provide tools for understanding the mechanisms of interleukin-1β-MyD88 mediated neuroinflammation and/or tauopathy.

### Compositions and Methods for Treatment

In one aspect, this disclosure describes compositions and methods for prophylactic and/or therapeutic treatment of tauopathy. Specifically, this disclosure describes a highly efficient, safe, economical, and state-of-the-art immunotherapy approach based on a Virus-Like Particle (VLP)-platform to target four disease-related modifications in MAPT as a potential therapy for MAPT pathology induced by traumatic brain injury (TBI). This strategy is not only relevant to TBI-induced MAPT pathology, but also can be applicable to other neurodegenerative tauopathies such as, for example, Alzheimer's disease and related tauopathies.

Pathological misfolding and aggregation of microtubule-associated protein tau (MAPT) into neurofibrillary tangles (NFTs) is a neuropathological hallmark of several neurodegenerative tauopathies. In addition, hyperphosphorylation and aggregation of MAPT occur following traumatic brain injury (TBI) and chronic traumatic encephalopathy (CTE), either or both of which can occur in athletes following repeated concussions.

Consequences of axonal injury following brain trauma include, for example, the release of MAPT into the interstitial fluid, the pathological alterations of MAPT, and/or the neuron-neuron propagation of pathological MAPT between anatomically connected neural networks as well as destabilization of microtubules. Although MAPT has been targeted in other devastating movement disorders with pure tauopathy, like progressive supranuclear palsy (PSP), no attempts have been made to target pathological MAPT in traumatic brain injury. Because MAPT trials have thus far failed to demonstrate efficacy, and because MAPT pathology is also relevant in age-related tauopathies, there is an urgent need to develop highly efficacious MAPT-targeted therapies to reverse the clinical course following traumatic brain injury.

One promising therapeutic strategy currently being tested in Alzheimer's disease is immunotherapy. There are several challenges with this approach. First, it is often difficult to elicit strong antibody responses against self-antigens like MAPT. Second, immune responses against self-antigens have the potential to cause substantial side effects (e.g., in the case of Aβ vaccination, they can lead to encephalitis, vasogenic edema, and/or microhemorrhage in 5% of immunized patients). Furthermore, while the administration of purified MAPT antibodies (passive immunization) shows promising trends in reducing pathological MAPT in recent studies, the therapy is not cost-effective due to the prohibitively expensive process of making purified antibodies. Finally, in the context of MAPT pathology, neuroinflammation, which can ensue in response to brain trauma, accelerates MAPT pathology and cognitive impairment in an hTau mouse model of tauopathy (Bhaskar et al., 2010, Neuron 68:19-31). It is, therefore, important to develop a MAPT-targeted immunotherapy that can successfully elicit a targeted immune response against neurofibrillary tangles while limiting the inflammatory consequences.

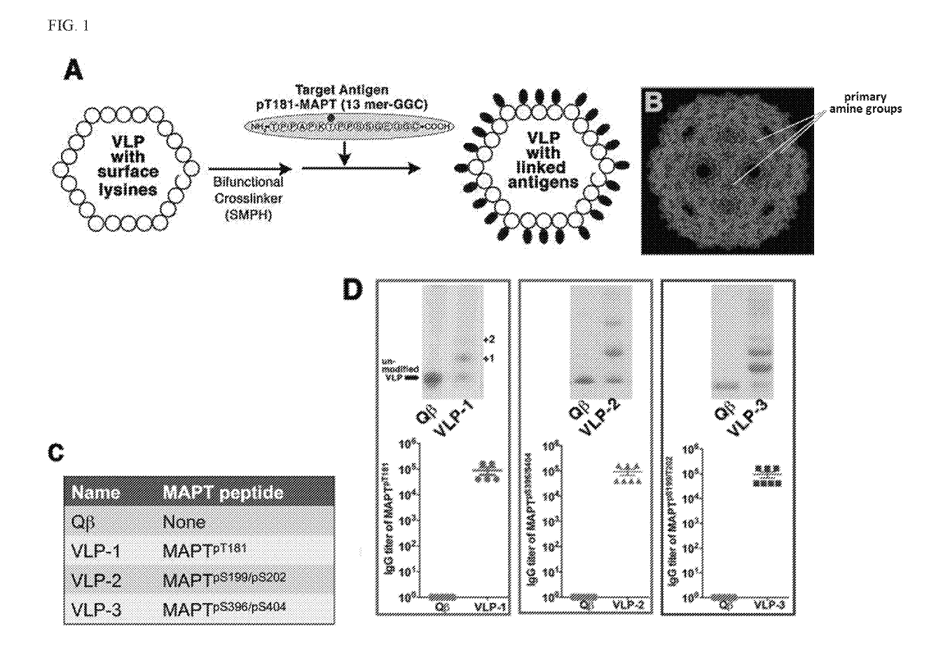

This disclosure describes exploiting a unique VLP-based platform technology to develop vaccines against tauopathies. This strategy uses non-infectious VLPs to display antigens at high valency on the surface of a highly immunogenic particulate antigen (FIGS. 1A and 1B). This disclosure further describes conjugating a peptide hyperphosphorylated at T181 (MAPT^{pT181} or pTau), S199/S202 (MAPTP^{S199/S202} and/or S196/S404 (MAPT^{pS396/S404}) to VLP derived from Qβ RNA bacteriophage (FIG. 1A-1D). MAPT^{pT181} is an early stage, disease-associated MAPT peptide and its presence in cerebrospinal fluid (CSF) has been used to diagnose Alzheimer's disease (Vanderstichele et al., 2006, Clin Chem Lab Med 44:1472-1480). MAPTP^{S199/S202} and MAPT^{pS396/S404} are early-stage and late-stage markers of MAPT pathology.

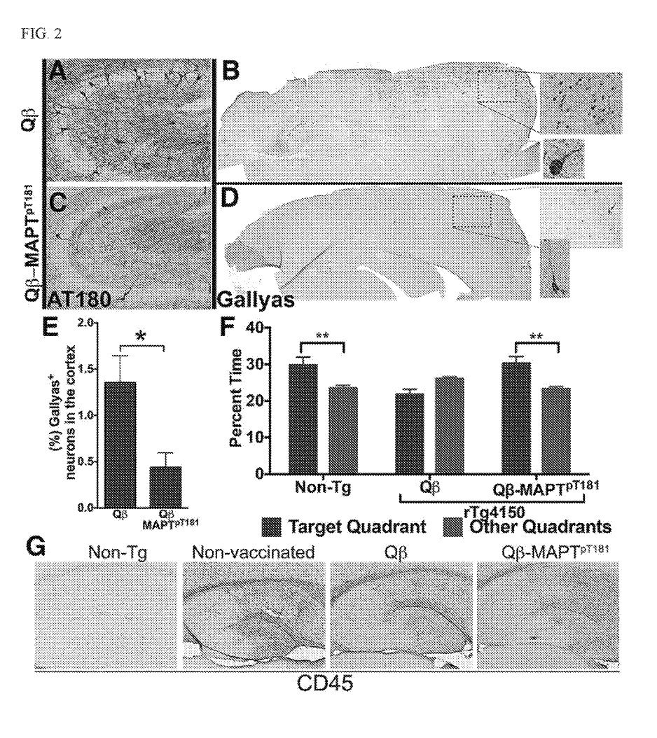

Immunizations with Qβ-MAPT^{pT181}, Qβ-MAPT^{pS199/S202}, and Qβ-MAPT^{pS396/S404} successfully elicited higher antibody titers (>10⁵ geometric mean antibody titers) than conventional active immunotherapy (FIGS. 1C and 1D). Thus, VLP-display of self-antigens can overcome the mechanisms of immunological tolerance. Notably, Qβ-MAPT^{pT181} immunotherapy reduced neurofibrillary tangle pathology (FIG. 2A-E) and improved spatial memory in the Morris Water Maze (MWM) test (FIG. 2F) without inducing unwanted T_{H}1-cell responses or neuroinflammation (FIG. 2G) in a rTg4510 transgenic mouse model of tauopathy. These results complement a recent study on the direct infusion of anti-MAPT antibodies, which was shown to clear pathological MAPT and improve cognitive function in a mouse model of tauopathy. Taken together, an antibody-mediated approach to block the onset and propagation of neurofibrillary tangle pathology following traumatic brain injury represents a feasible therapeutic approach.

Thus, this disclosure describes, in one aspect, a viable, safe, and comprehensive immunotherapy approach to treat and, in some cases, reverse MAPT pathology, neurodegeneration, and/or cognitive decline in response to traumatic brain injury or in neurodegenerative tauopathies such as Alzheimer's disease. In some cases, neurodegeneration can include brain atrophy. In some cases, the immunotherapy also can minimize undesirable inflammatory response and possible targeting of non-pathological MAPT. As used herein, “treat,” “treatment,” and variations thereof may be therapeutic or prophylactic. Therapeutic treatment typically is initiated after a subject exhibits one or more symptoms or clinical signs of a condition. “Symptom” refers to any subjective evidence of disease or of a patient's condition. “Sign” or “clinical sign” refers to an objective physical finding relating to a particular condition capable of being found by one other than the patient. Prophylactic treatment typically is initiated before a subject exhibits one or more symptoms or clinical signs of a condition. Prophylactic treatment may be initiated when a subject is considered “at risk” of developing a given condition. As used herein, the term “at risk” refers to a subject that may or may not actually possess the described risk. Thus, for example, a subject “at risk” of a condition is a subject possessing one or more risk factors associated with the condition such as, for example, genetic predisposition, ancestry, age, sex, geographical location, lifestyle, or medical history.

Generally, the treatment involves administering to a subject having or at risk of having a condition associated with tauopathy a composition that includes an antigen presentation component and/or immunogenic component linked to a MAPT component. In some embodiments, the antigen presentation component and/or immunogenic component preferably includes a virus-like particle (VLP)-based immunogenic component and/or a virus-like particle (VLP). In some embodiments, the MAPT component includes a MAPT antigen epitope.

In some embodiments, a VLP-based immunogenic component may be conjugated to at least one of the four disease-associated MAPT peptides as antigen epitopes, designated herein as MAPT^{pS199/S202}, MAPT^{pS396/S404}, MAPT^{pT181}, and MAPT^{ΔD421} (Table 1).

TABLE 1
Exemplary MAPT peptides for use in VLP therapy

| Peptide | Source | Characteristics |
|---|---|---|
| MAPT^{ΔD421} (SEQ ID NO: 5) | Plasmid/bacterial | Caspase-3 or caspase-7 cleaved MAPT involved |
| | expression | in aggregation process and precedes |
| | | hyperphosphorylation events. Prevalent in |
| | | Alzheimer's disease brain tissues and inversely |
| | | correlates with cognitive function. |
| MAPT^{pS199/S202} (SEQ ID NO: 2) | Custom synthesis | S199/S202 are the first residues to be |
| | | hyperphosphorylated during disease progression |
| | | and produces the epitope for the antibody AT8. |
| MAPT^{pT181} (SEQ ID NO: 1) | Custom synthesis | Phosphorylated T181 is an epitope for the |
| | | antibody AT270. MAPT^{pT181} is a CSF biomarker |
| | | for diagnosis of Alzheimer's disease. |
| MAPT^{pS396/S404} (SEQ ID NO: 4) | Custom synthesis | Phosphorylated S396/S404 are the epitopes for |
| | | the antibody PHF1. PHF1 reacts to more mature |
| | | hyperphosphorylated forms of MAPT found |
| | | primarily in late-stage tangles. |

In some embodiments, a VLP-based immunogenic component may be conjugated to a MAPT component where the MAPT component includes the amino acid sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33.

In some embodiments, the MAPT component includes at least one amino acid residue modified to include a PO₃H₂ group.

In some embodiments, a VLP-based immunogenic component may be conjugated to a MAPT peptide where the MAPT peptide is identified using phage display. For example, antibodies which bind to known MAPT peptides may be identified using ELISA and/or affinity selection. These antibodies may then be used to select additional MAPT peptides displayed by a phage library including, for example, a MAPT peptide with mutations and/or deletions, a peptide portion of an unmutated MAPT peptide, or a peptide portion of a MAPT peptide with mutations and/or deletions.

In some embodiments, the VLP may serve as a non-self, surrogate antigen for a conformation-specific MAPT antibody (MC1-early stage; Jicha et al., 1997, J Neurosci Res 48: 128-132) that is specific to pre-tangle conformation. VLPs directly displaying truncated and/or hyperphosphorylated MAPT in a multivalent pattern can lead to strong immunogenicity. Furthermore, VLPs reactive to MC1 antibody can serve as mimics of conformational MAPT epitopes and non-self and surrogate immunogens.

In a first embodiment, a VLP (e.g., a Qβ bacteriophage VLP) can be chemically conjugated to a pathological MAPT peptide through either N-terminal or C-terminal cysteine residues. One can use a mouse model to immunize normal mice with the conjugated VLPs and measure antibody responses to the targeted peptides (e.g., by ELISA). While other bacteriophage VLPs may be used, Qβ VLPs are composed of a single coat protein that self-assembles into a 27 nm-diameter icosahedral particle consisting of 90 coat-protein dimers. Moreover, MAPT peptides containing single free cysteine residues easily link to primary amine groups on surface-exposed lysines on VLPs via a bi-functional cross-linker with amine- and sulfhydryl-reactive arms. These residues allow conjugation of several MAPT peptides per VLP molecule, increasing the antibody response that is induced, and overcoming possible immune tolerance against a self-antigen like MAPT.

### Conjugating MAPT to VLPs

MAPT peptide can be covalently linked to a VLP. For example, a MAPT peptide containing an N-terminal or C-terminal cysteine residue can be linked to VLPs (e.g., Qβ VLPs, MS2, PP7, AP205, or any phage in the Leviviridae family) using a bi-functional cross-linker as illustrated in FIG. 6. One exemplary suitable bi-functional cross-linker is succinimidyl 6-((beta-maleimidopropionamido)hexanoate) (SMPH). MAPT^{pS199/S202} (SEQ ID NO:2), MAPTP^{T181} (SEQ ID NO:1), and MAPT^{pS396/S404} (SEQ ID NO:4) are custom-synthesized peptides from commercial sources. These peptides are designed to incorporate phosphorylated S/T residues that are spaced approximately at the center of the peptide chain. In some embodiments, the peptides are engineered to include a cysteine residue at the N-terminus for easy conjugation with the free lysines of the VLPs. For MAPT^{ΔD421}, a stop-codon is inserted at Asp421 in a full-length human MAPT construct. One can insert a cysteine (linker) at the N-terminus of the protein for VLP conjugation using site-directed mutagenesis. One can express this truncated and genetically modified version of MAPT^{ΔD421} in, for example, E. coli and purify the protein as previously described (Bhaskar et al., 2005, J Biol Chem 280:35119-35125). The extent of conjugation of MAPT to Qβ VLPs can be analyzed by SUS-PAGE (see, e.g., FIG. 1D), Western blot analysis with respective immunized sera, and/or ELISA (FIG. 1D). Following production of MAPT-VLPs, immunogenicity can be assessed by immunizing C57Bl/6 mice using previously described protocol (Chackerian et al., 2006, Vaccine 24:6321-6331).

While occasionally described in the context of exemplary embodiments in which the MAPT peptide is displayed using a VLP, an immunogen as described herein may be constructed, a composition as described herein may be prepared, and a method as described herein may be practiced using any suitable platform for displaying the MAPT peptide. Suitable platforms for displaying an immunogenic peptide include, for example, any synthetic and/or biocompatible platform that can display an immunogenic peptide in a multivalent format and/or array for presentation to the immune system. Such platforms can involve the use of a virus, a virosome, and/or nanoparticles.

### Developing MAPT-VLP Based Intervention Against TBI-Induced Tau Pathology

Among several changes, neuroinflammation and MAPT hyperphosphorylation are primary consequences of traumatic brain injury and chronic traumatic encephalopathy. It is therefore necessary to determine the appropriate therapeutic window where VLP-MAPTs can efficiently clear hyperphosphorylated MAPT following traumatic brain injury and prevent further seeding/propagation.

Two-month old non-transgenic (WT) and hTau mice can be subjected to Single Air-blast Injury. The mice can be subjected to MRI at 1-day, 3-days, 14-days and 120-days post-injury (DPI) and then sacrificed for biochemical and neuropathological analysis. Mice in the 14-DPI and 120-DPI window can be subjected to behavioral analysis prior to Mill and euthanasia. The extent of MAPT pathology may correlate with cognitive decline in several age-related tauopathies and following fluid percussion injury. Moreover, enhanced neuroinflammation can accelerate MAPT pathology and cognitive impairment in a mouse model of tauopathy. Thus, optimal treatment for recovery and repair for immunotherapy can involve treatment during the optimum therapeutic window.

Indeed, a significant number of inflammatory components are altered in Alzheimer's disease and related tauopathies. For example, gliosis (increased number of microglia and astrocytes) and elevated levels of numerous inflammatory molecules are observed in human brain tissue of Alzheimer's disease patients as well as in the brains of amyloid-independent tauopathies (e.g., progressive supranuclear palsy (PSP), corticobasal degeneration (CBD), and Pick's disease (PiD)). As another example, long-term treatment with NSAIDs reduced the risk of developing Alzheimer's disease by >50%. Moreover, alterations in inflammatory cells/molecules prior to MAPT aggregation have been observed in several different mouse models of tauopathy. Finally, enhancing microglial activation via LPS exacerbated MAPT pathology in 3×Tg and rTg4510 mouse models of tauopathy, while blocking microglial activation via an immunosuppressant drug (FK506) attenuated MAPT pathology and extended the life span of the P301S mouse model of tauopathy. Together, these observations indicate that neuroinflammatory processes may be involved in and/or regulate tangle pathology.

Numerous studies have attempted to develop active or passive immunotherapy strategies against hyperphosphorylated and/or misfolded MAPT. First, active immunization with AD-specific phosphorylated MAPT epitope reduce level of aggregated MAPT in the brain and slowed the progression of the tangle-related behavioral phenotype in the JNPL3 mouse model of pure tauopathy. Second, clearance of extracellular MAPT aggregates reduces neurodegeneration and prevents the trans-synaptic spread of MAPT pathology. Finally, antibodies to misfolded MAPT, similar to those for α-synuclein, can enter the brain, bind to pathological MAPT within neurons, reduce MAPT aggregates, and prevent cognitive decline. While these studies clearly suggest that intracellular misfolded proteins such as MAPT or α-synuclein can serve as targets for immunotherapeutic approaches, these methods remain susceptible to inducing excessive neuroinflammation, which may exacerbate MAPT pathology and result in unanticipated side effects.

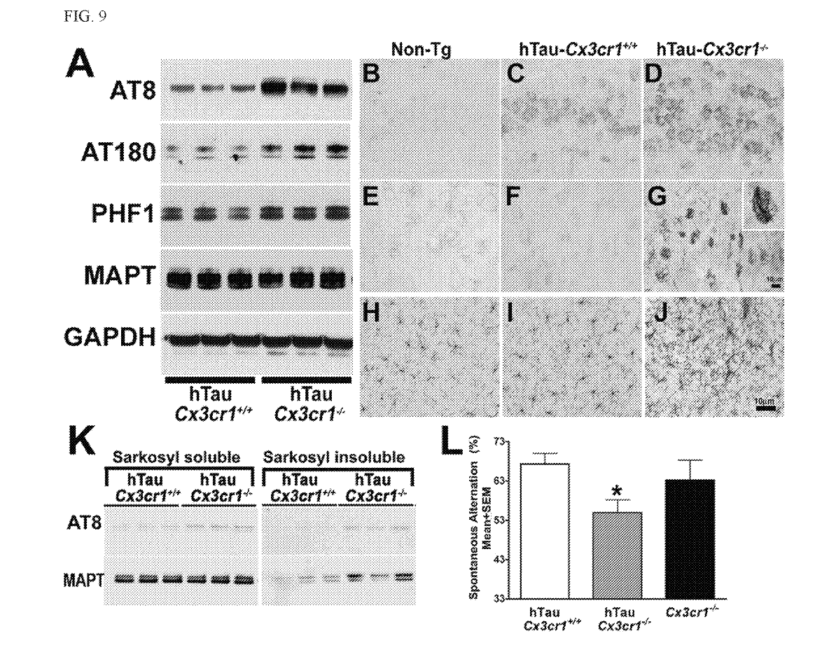

hTau mice display age-dependent microglial activation and, which temporally correlates with the progression of MAPT phosphorylation and aggregation. Also, hTau mice displayed significantly increased levels of soluble fractalkine (CX3CL1) at the time of rapid MAPT phosphorylation and aggregation. CX3CL1 is the only ligand for fractalkine receptor, CX3CR1 that is exclusively expressed by microglia within the CNS and is important for negative regulation of microglial activation. Genetic deficiency of CX3CR1 in hTau mice results in elevated microglial activation, accelerated MAPT pathology, and impairments in working memory (FIG. 9).

Immunizing Injured hTau Mice with Engineered MAPT-VLPs

VLP-based targeted immunotherapy against pathological MAPT can elicit a robust immune response against a self-antigen like MAPT, significantly reduce MAPT burden, and/or improve cognitive function in one of the most pathologically aggressive mouse models of tauopathy (rTg4510) (FIG. 2). The efficacy of immunizing a subject having or at risk of having a condition associated with tauopathy with a VLP-based component that includes a MAPT antigen epitope can be established using a mouse model. For example, hTau mice subjected to SAI can be administered compositions that include either MAPT-VLPs. The mice can be evaluated to determine whether the compositions reduce or limit the progression of the disease, block/clear MAPT hyperphosphorylation and aggregation, improve cognitive function, and/or alter the clinical course of the disease.

MAPT-VLPs (e.g., MAPT^{ΔD421} (SEQ ID NO:5), MAPT^{pS199/S202} (SEQ ID NO:6), and MAPT^{pS396/S404} (SEQ ID NO:4)) can elicit different degrees of antibody response in hTau mice. Nonetheless, these VLPs can target and clear respective human MAPT antigens in the immunized hTau mouse brains following SAI.

Inoculation of SAI-hTau mice with MAPT-VLPs can elicit an antibody response specific to the respective antigens. Thus, administering the MAPT-VLPs significant can reduce the level of respective pathological MAPT in biochemical and neuropathological analysis. This VLP-strategy can improve SAI-induced behavioral impairment and/or structural deficits compared to control groups. Neuroinflammation due to the VLP vaccinations, which is unlikely given preliminary observations that the brains of MAPT-pT181-VLP-vaccinated mice have reduced CD45+ microglial inflammation (FIG. 2G), can be supplemented with anti-inflammatory strategies as described in Table 2. Alternatively, one can increase the stoichiometric ratio of MAPT:VLPs and/or supplement the MAPT-VLP with an alternative adjuvant, similar to those utilized in Aβ immunization studies (Chackerian et al., 2006, Vaccine 24:6321-6331).

TABLE 2
Exemplary strategies to block T_{H}1 cell response and/or avoid excessive
neuroinflammation

| | Strategy | Rationale | Approach |
|---|---|---|---|
| 1 | Enriching | IgG4 backbone (e.g., crenezumab) | Co-injections of MAPT-VLPs with |
| | immunoglobulins, | binds less weakly to cell surface | Merck Aluminum Adjuvant (MAA) |
| | e.g., with an enhanced | IgG receptors and therefore has | will be performed since VLPs |
| | IgG4 (functionally | milder effects on activation of | formulated with MAA have been |
| | similar in mice to | microglia, safely promotes a T_{H}2 | demonstrated to promote IgG4- |
| | IgG3) backbone | cell response. | enriched and T_{H}2 immune response |
| | | | in HPV vaccination studies. |
| 2 | Removing RNA from | Removing RNA from VLPs | VLPs lacking endogenous RNA |
| | VLPs to promote T_{H}2 | enhances T_{H}2 cell response. | will be utilized for conjugation of |
| | response | | MAPT. This would preferentially |
| | | | promote T_{H}2 cell response. |
| 3 | Enrichment of | Although rare, B10 cells are | Co-injections of MAPT VLPs with |
| | regulatory B cells | potent negative regulators of | IL10 or IL21, to demonstrate this |
| | with the ability to | antigen-specific inflammation and | preferential enrichment of B 10 |
| | express the anti- | T-cell-dependent autoimmune | cells. |
| | inflammatory | disease in mice. | |
| | cytokine IL10 (B10 | | |
| | cells). | | |

Qβ-MAPT^{pT181}, Qβ-MAPT^{pS396/S404}, and Qβ-MAPT^{p199/S202} Vaccination Studies

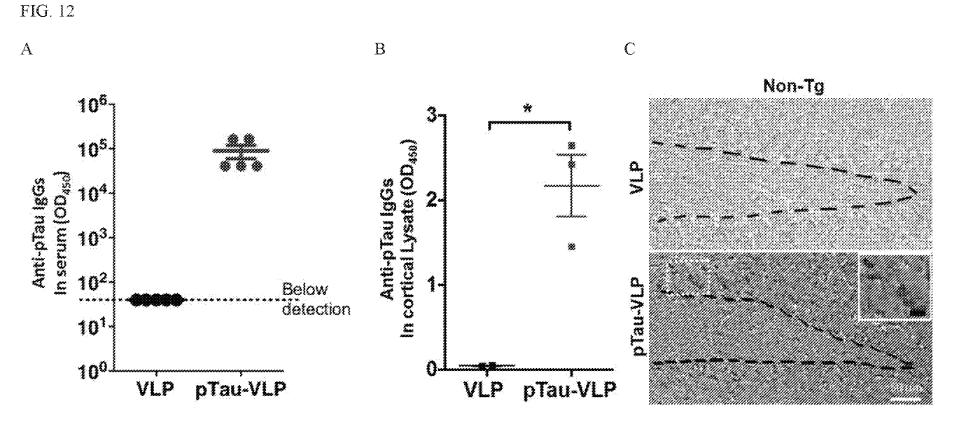

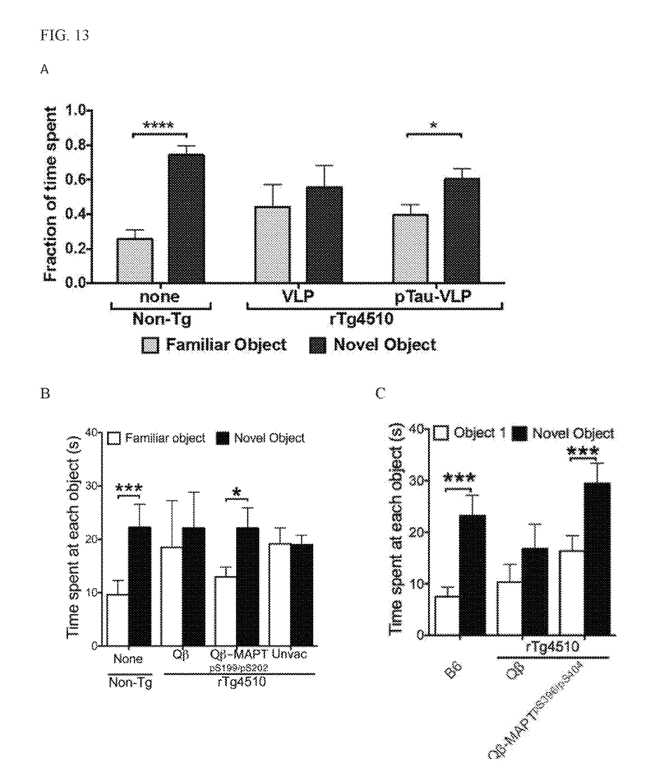

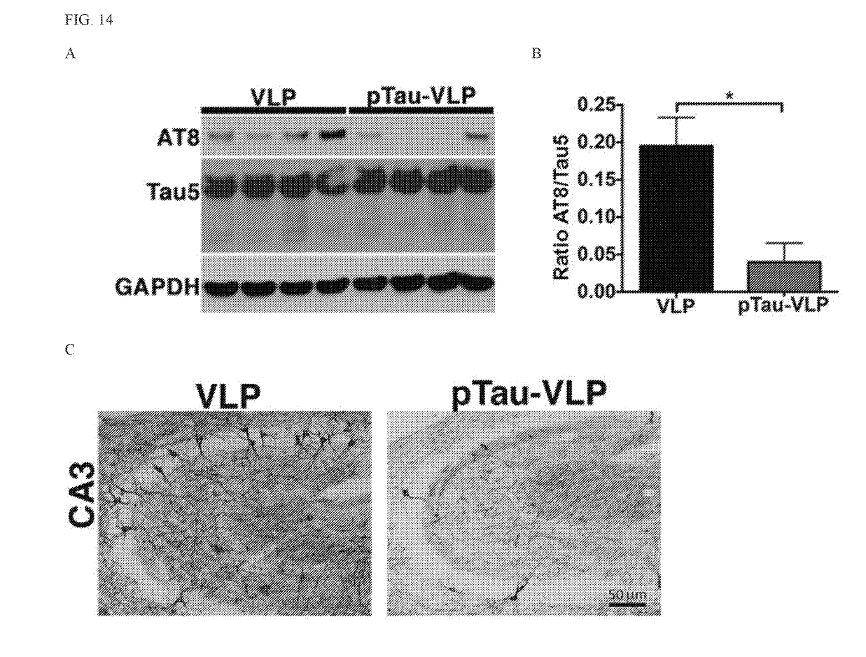

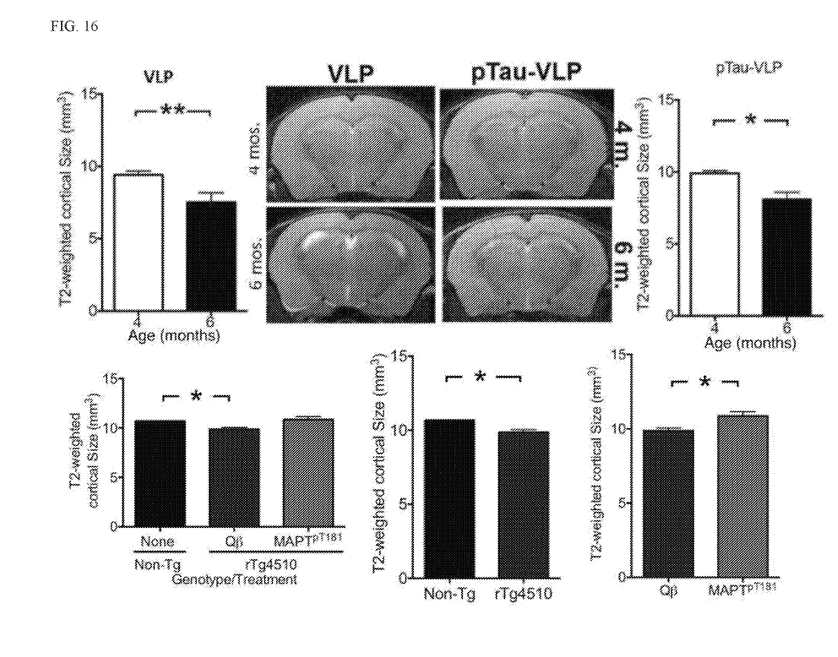

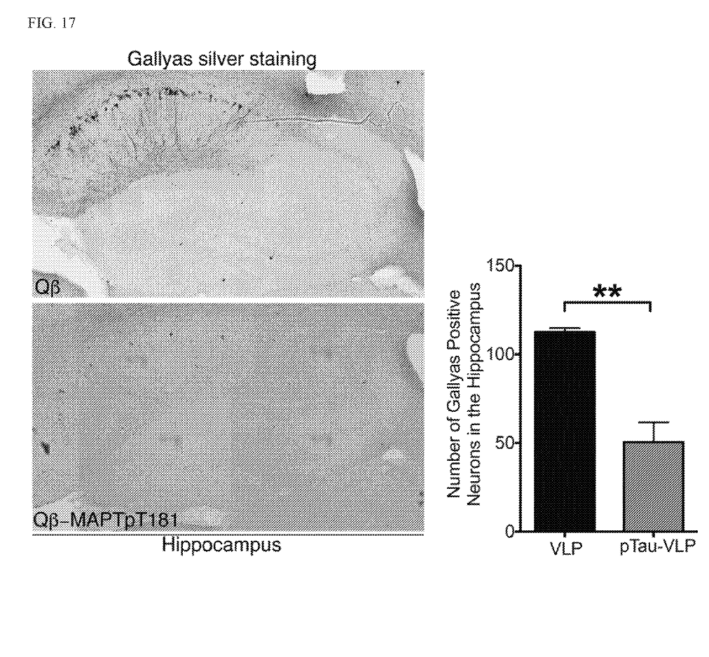

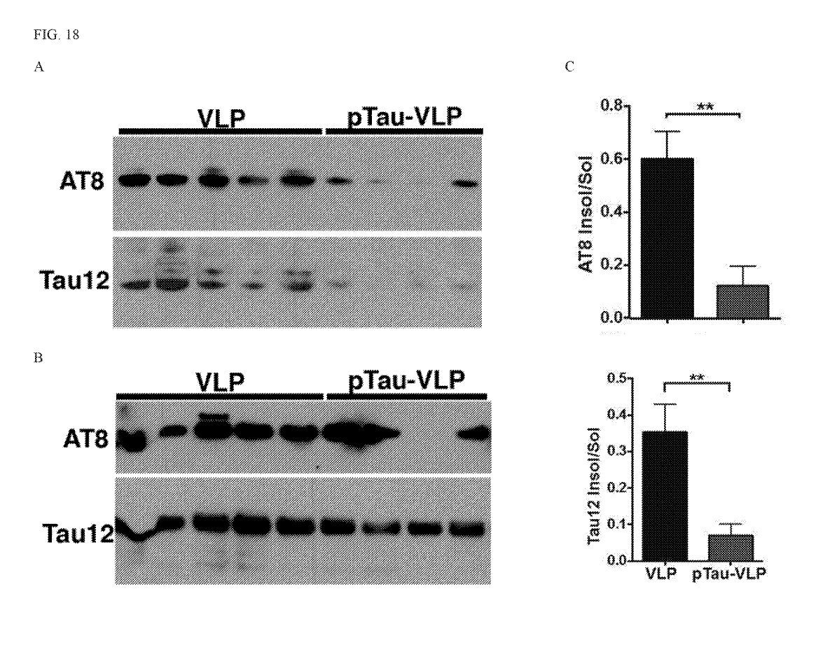

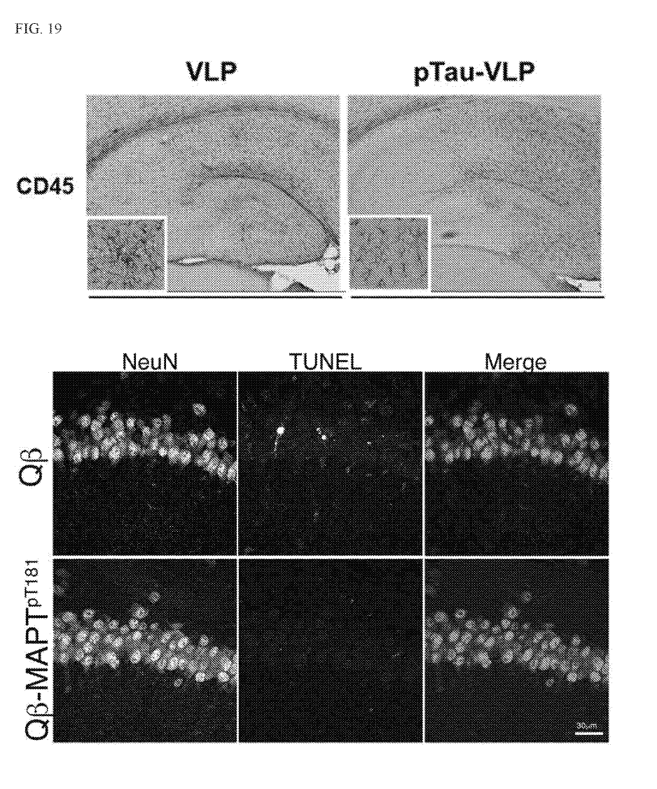

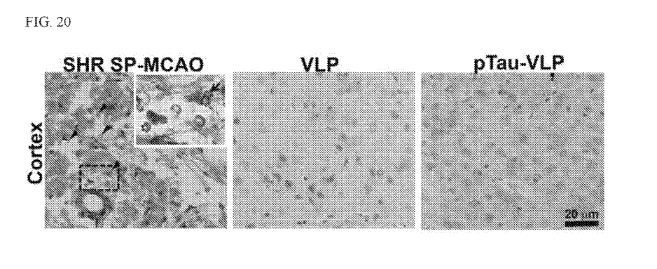

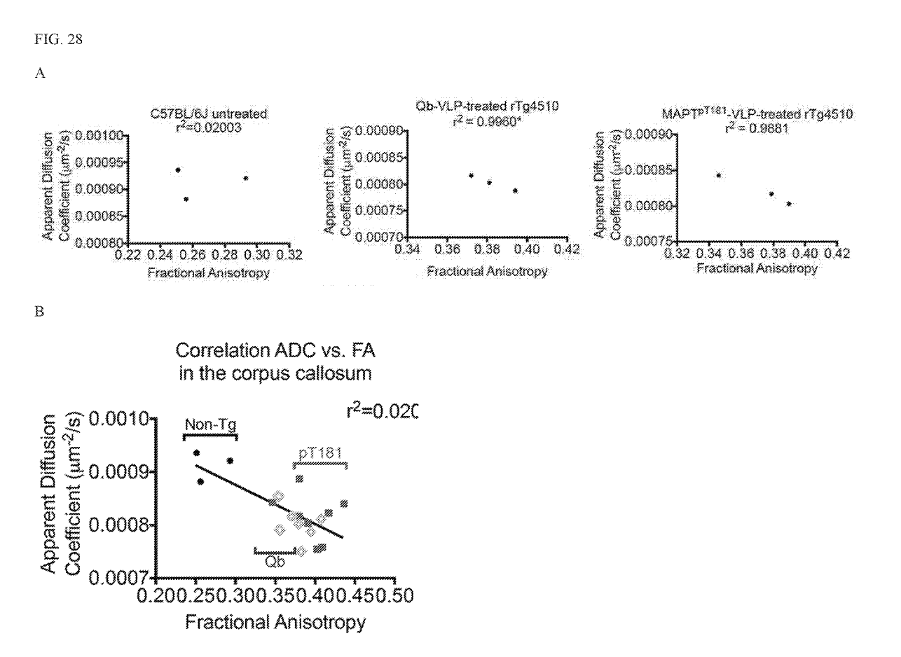

Although vaccinating with a VLP conjugated to MAPT (e.g., Qβ-MAPT^{pT181}) can generate a robust response, vaccinating with a MAPT peptide (e.g., MAPT^{pT181}) or Qβ alone generates an IgG response below the detection limit suggesting that conjugation of a VLP conjugated to MAPT is an important step in enhancing high-titer antibody response. Antibodies generated to MAPT^{pT181} after immunization with Qβ-MAPT^{pT181} can penetrate the brain (cortex) (FIG. 12), and rTg4510 mice vaccinated with Qβ-MAPT^{pT181} can display significantly reduced levels of AT8+ MAPT (FIG. 14), silver positive tangles (FIG. 17), insoluble/aggregated tau tangles (FIG. 18), brain atrophy (T2 weighted images, FIG. 15, FIG. 16), and white matter damage (ADC and FA correlation, FIG. 28) compared to injected rTg4510 mice. Mice vaccinated with Qβ-MAPT^{pT181} can also show reduced neurodegeneration/neuronal loss, as measured by immunofluorescence analysis (FIG. 19) but do not display any evidence of hemorrhage in the brain (FIG. 20).

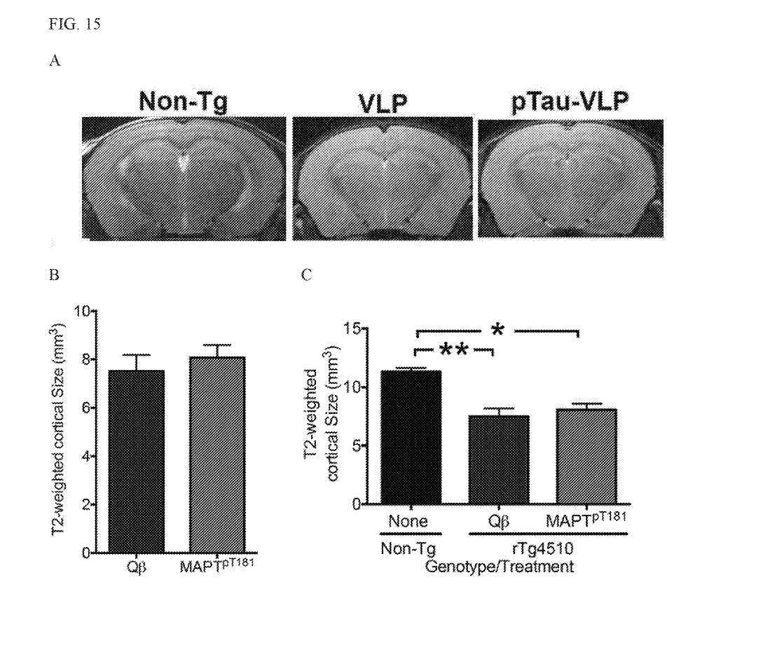

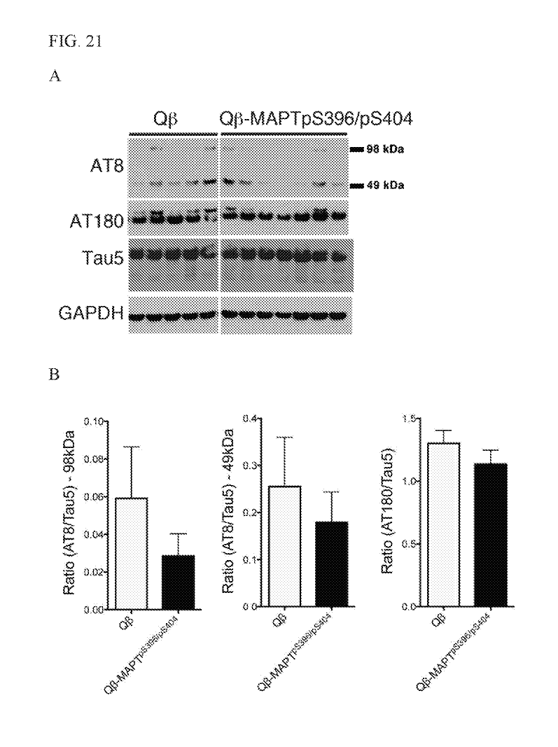

Vaccination with Qβ-MAPTP^{S396/S404} can reduce tau phosphorylation at AT8 (S199/S202) and AT180 (T231) sites (FIGS. 21 A, B). And Qβ-MAPT^{pS396/S404} and Qβ-MAPT^{pS199/S202} immunized mice can show improved recognition memory (FIG. 13B, C).

hTau Mouse Studies

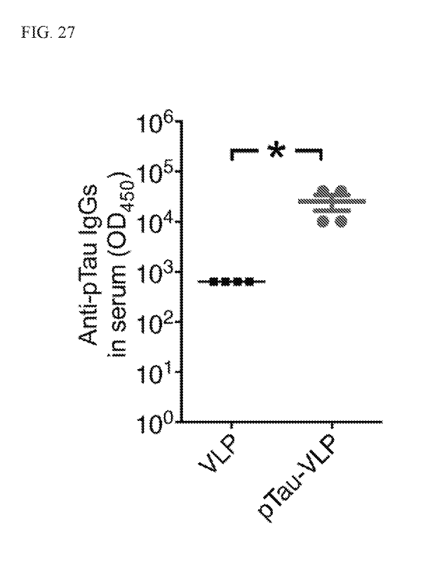

To test the effect of vaccination in a different mouse model of tauopathy, hTau mice were immunized with Qβ or Qβ-MAPT^{pT181} peptides. Similar to rTg4510 mice, statistically significant increased titers for anti-pTau IgG were observed in the sera of 6-month-old hTau mice after vaccination with MAPT^{pT181} peptide (pTau-VLP) compared to titers observed in mice vaccinated with Qβ alone (FIG. 27). These results suggest that in two different mouse models of tauopathy, Qβ-MAPT^{pT181} vaccination can result in significant IgG response.

A MAPT-VLP can, therefore, be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with MAPT-VLP without causing any undesirable biological effects or interacting in a deleterious manner with any of the other components of the pharmaceutical composition in which it is contained.

A MAPT-VLP may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, intrathecal etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

A formulation may be conveniently presented in unit dosage form and may be prepared by methods well known in the art of pharmacy. Methods of preparing a composition with a pharmaceutically acceptable carrier include the step of bringing the MAPT-VLP into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

A MAPT-VLP may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like. The formulations can be administered as a single dose or in multiple doses.

The amount of MAPT-VLP administered can vary depending on various factors including, but not limited to, the specific MAPT-VLP being administered, the weight, physical condition, and/or age of the subject, and/or the route of administration. Thus, the absolute weight of MAPT-VLP included in a given unit dosage form can vary widely, and depends upon factors such as the particular MAPT-VLP being administered, the species, age, sex, weight and physical condition of the subject, and/or the method of administration. Accordingly, it is not practical to set forth generally the amount that constitutes an amount of a MAPT-VLP effective for all possible applications. Those of ordinary skill in the art, however, can readily determine the appropriate amount with due consideration of such factors.

In some embodiments, the method can include administering sufficient MAPT-VLP to provide a dose of, for example, from about 100 ng to about 50 mg to the subject, although in some embodiments the methods may be performed by administering MAPT-VLP in a dose outside this range. In some of these embodiments, the method includes administering sufficient MAPT-VLP to provide a dose of from about 10 μg to about 5 mg to the subject, for example, a dose of from about 100 μg to about 1 mg. In one specific embodiment, the method includes administering sufficient MAPT-VLP to provide a dose of from about 25 μg to about 300 μg.

Alternatively, the dose may be calculated using actual body weight obtained just prior to the beginning of a treatment course. For the dosages calculated in this way, body surface area (m²) can be calculated prior to the beginning of the treatment course using the Dubois method: m²=(wt kg^{0.425}×height cm^{0.725})×0.007184.

In some embodiments, the method can include administering sufficient MAPT-VLP to provide a dose of, for example, from about 0.01 mg/m² to about 10 mg/m².

In some embodiments, a MAPT-VLP may be administered, for example, from a single dose to multiple doses per week, although in some embodiments the method can be performed by administering the MAPT-VLP at a frequency outside this range. In certain embodiments, the MAPT-VLP can be administered on an as needed basis.

### Mouse Model

In another aspect, this disclosure also describes a new mouse model that can provide tools for understanding the mechanisms of neuroinflammation and/or tauopathy. The model involves hTau mice that express all six isoforms of non-mutant human tau, in the endogenous mouse tau knockout background.

Tangle pathology (including hyperphosphorylated and aggregated tau) is a neuropathological sign of Alzheimer's disease and related tauopathies. Inflammatory processes in the brain typically precede tau pathology. For example, enhancing neuroinflammation either via a Toll-Like Receptor-4 (TLR-4) ligand lipopolysaccharide (LPS) or genetic ablation of the Cx3cr1 (fractalkine receptor) lead to increased tau phosphorylation, aggregation, and working memory impairment in a manner dependent upon the activation of interleukin-1 receptor (IL-1R)-p38 mitogen activated protein kinase (p38 MAPK) pathway.

Also, activating the microglial interleukin-1β (IL-1β) signaling pathway may drive neuroinflammation and cause tau pathology, while blocking IL-10 signaling can prevent tau pathology and neuronal damage. Thus, while inflammatory alterations in microglia can precede tau pathology and lead to neurodegeneration, the factor(s) driving neuroinflammation in tauopathies are unknown. When human tau is overexpressed in neurons, it undergoes disease-related hyperphosphorylation and is secreted into the extracellular space where it activates microglia or macrophages to induce IL-1β-mediated neuroinflammation. Moreover, inflammatory molecules are upregulated at early disease stages in hTau mice. Thus, pathological tau can induce neuroinflammation.

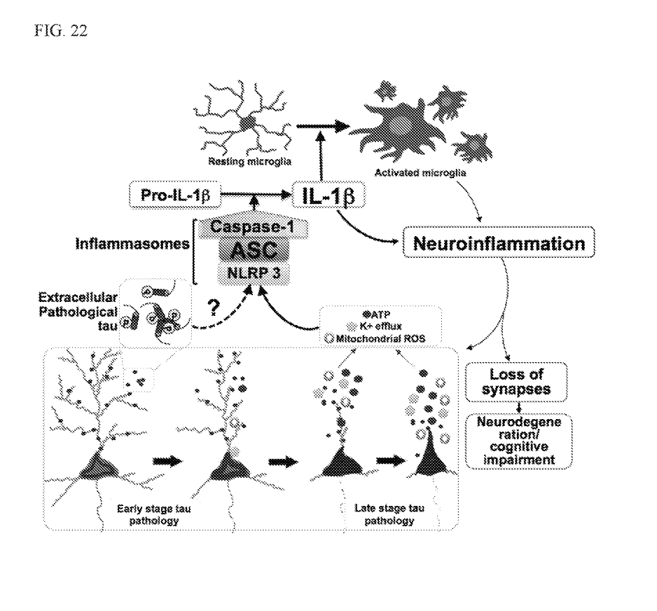

One possibility is that pathological tau engages the inflammasome complex (a sensor of cellular stress) to drive chronic low-grade brain inflammation in tauopathies. (FIG. 22). For example, genetically blocking pathological tau or IL-1β activation may delay the disease progression of tauopathy by preventing neuronal loss and cognitive impairment. If the mechanisms of neuroinflammation and tauopathy are better understood, the development of therapies for neurodegenerative diseases that target the inflammasome may be accelerated. Tauopathies are characterized by altered inflammatory components. First, gliosis and increased inflammatory molecules are observed in the brains of both Alzheimer's and non-Alzheimer's tauopathies (e.g., progressive supranuclear palsy (PSP)). Second, in a retrospective epidemiological study, long-term treatment with NSAIDs reduced the risk of developing Alzheimer's disease by >50%. Third, altered inflammation precedes tau aggregation in several different mouse models of tauopathy (FIG. 22). Fourth, enhancing microglial activation through lipopolysaccharide (LPS) exacerbated tau pathology in 3×Tg and rTg4510 mouse models of tauopathy. Correspondingly, blocking microglial activation with an immunosuppressant drug (e.g., FK506) attenuated tau pathology and extended the life span in a P301S mouse model of tauopathy. Fifth, recent genome-wide association studies have identified single-nucleotide polymorphisms in key inflammatory genes, including a complement component (3b/4b) (CR1 locus) and an R47H substitution in the TREM2 gene behind the development of late-onset (sporadic) Alzheimer's Disease. Taken together, these results suggest that immune/inflammatory pathways actively contribute to the development of Alzheimer's disease and related dementias.

Microglia are the resident macrophages forming the first line of defense in the brain. One feature of microglial cells is their rapid activation in response to even minor pathological insults in the central nervous system. In many neurodegenerative diseases, aggregation of misfolded protein (e.g., Aβ in Alzheimer's disease and α-synuclein in Parkinson's disease) can act as a potent pro-inflammatory trigger for activation and subsequent secretion of numerous cytokines and chemokines from microglial cells. Therefore, inhibiting microglial recruitment and activation can help restore homeostasis and limit host tissue damage.

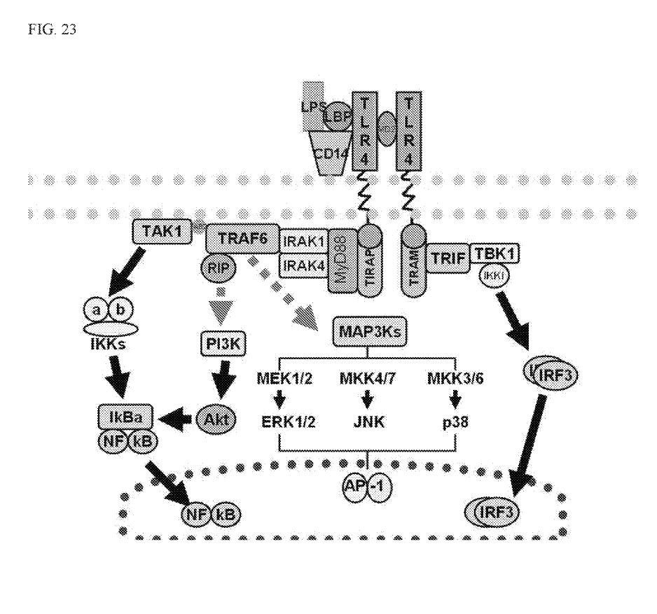

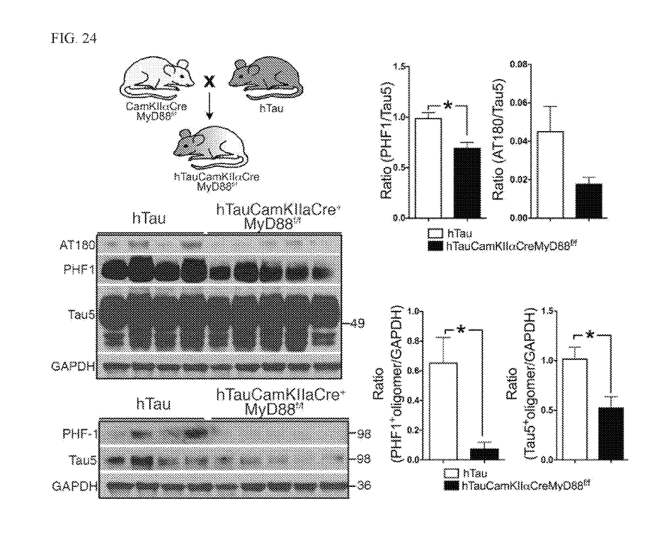

As mentioned above, inflammatory processes in the brain typically precede tau pathology. To block IL-1R and TLR-4 receptor signaling, MyD88—a common adapter protein of IL-1 and TLR-4 (FIG. 23) receptors—was targeted and deleted in a cell-specific fashion using Cre-LoxP technology. This disclosure describes a transgenic mouse model that allows one to genetically block MyD88 expression. MyD88 is an adapter protein involved in IL-1β activation in hTau mice. hTau mice were crossed to CamK2αCreMyD88^{f/f} mice (which express Cre recombinase in the forebrain neurons and include two copies of floxed MyD88 alleles). Neuron-restricted deletion of MyD88 reduces soluble (50 kDa) total tau (Taus) and tau hyperphosphorylated at PHF1 and AT180 sites (FIG. 24). It also reduces oligomeric (98 kDa) PHF1 positive tau in the hippocampus of 6-month-old hTauCamK2αCreMyD88^{f/f} mice compared to 6-month old hTau mice (FIG. 24). Notably, hyperphosphorylation of tau on Thr231 (recognized by the antibody AT180) and Ser396/Ser404 (recognized by the antibody PHF-1) are common pathological modifications that manifest in tau early in the disease process. Significantly lower amount of phosphorylated Thr231 and Ser396/Ser404 were observed in the hTauCamK2αCreMyD88^{f/f} mice compared to age-matched hTau mice via Western blot analysis. Tau oligomers dimers with a molecular weight of 100 kDa can be neurotoxic and/or can impair memory in mice. Significantly reduced levels of tau oligomers in hTauCamK2αCreMyD88^{f/f} mice compared to age-matched hTau mice were observed via Western blot analysis.

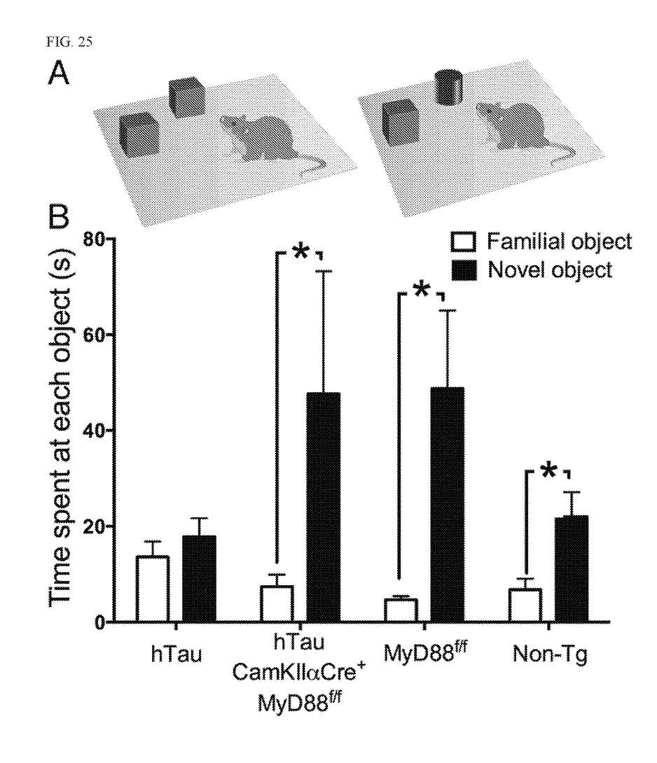

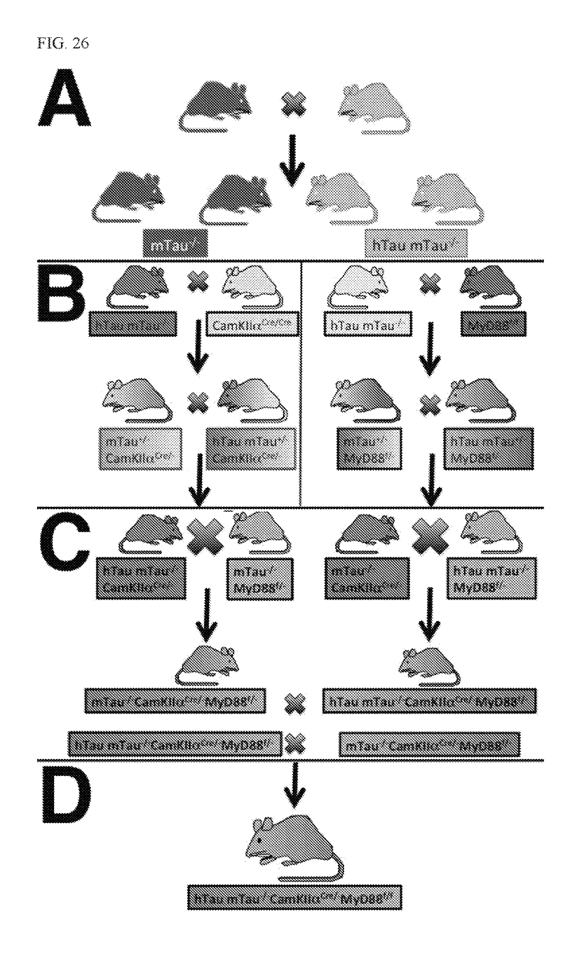

The novel object recognition (NOR) test was used to assess recognition memory in hTauCamK2αCreMyD88^{f/f} and hTau mice. Reduction of p-Tau levels improves recognition memory assessed via novel object recognition test (FIG. 25). The NOR test involves three days of behavioral testing. Briefly, on day one, animals are placed in an open arena (60 cm×50 cm×40 cm) for 10 minutes of habituation. On day two, animals are placed in the same open arena and exposed to two similar objects for 10 minutes (FIG. 25A). On day three, animals are placed in the same open arena and exposed to one familiar object (from day two) and one novel object (distinct in shape and texture from the familiar object) for 10 minutes (FIG. 25A). Mice with intact recognition memory spend more time exploring the novel object compared to the familiar object on the day three. However, if mice are impaired in recognition memory, they spend equal amount of time with both familiar and novel objects. As shown in the FIG. 25B, hTau mice spend equal time with both novel and familiar objects on day three of testing. However, hTauCamK2αCreMyD88^{f/f} mice spent more time exploring novel object similar to control groups of mice (FIG. 25B), suggesting improved memory in hTauCamK2αCreMyD88^{f/f} mice. In summary, neuron-restricted deficiency of MyD88, which blocks interleukin-1 receptor signaling specifically in neurons, reduces tau pathology and improves recognition memory in hTau transgenic mouse model of tauopathy.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1—Compression-Driven Shock Tube Model of Single Air Blast Injury (SAI)

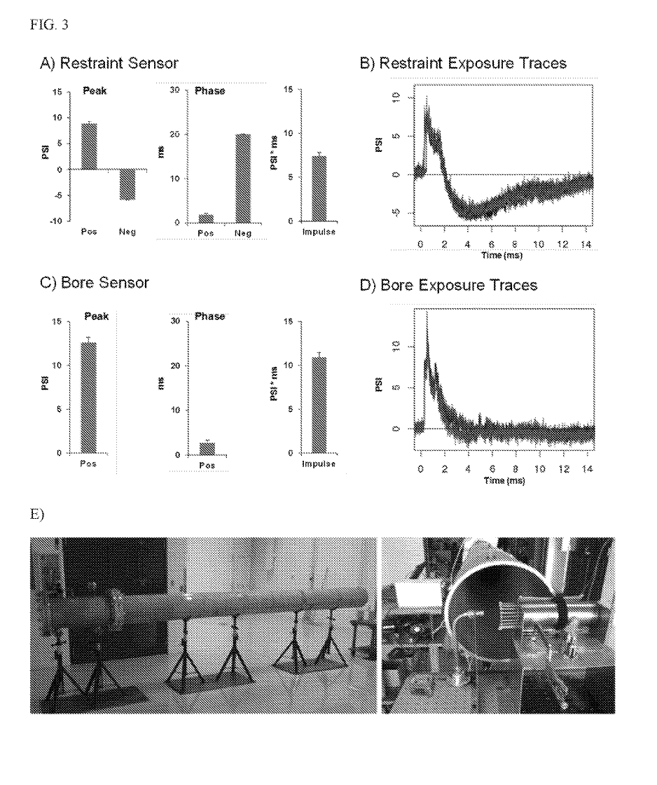

The Shock Wave Facility at Lovelace Respiratory Research Institute (LRRI) is located at the Kirtland Air Force Base in Albuquerque, N. Mex. The LRRI shock tube consists of a 12-inch diameter×2.5 ft long compression chamber and a 12-inch diameter×15 foot long expansion chamber (FIG. 3E). The chambers are separated by a Polyethylene Terephtalate (PET) Mylar (DuPont, Inc., Wilmington, Del.) sheet, which serves as the rupture point. This design is similar to the Shock Tube at Walter Reed Army Institute of Research, as previously reported (Long et al., 2009, J Neurotrauma 26:827-840). A trigger controlled by a solenoid valve is used to rupture the sheets. Multiple mylar sheets can be used for higher rupture pressures. The sheet holder is placed between the two chambers with PTFE gaskets on both sides. Piezoelectric free field blast probes (Piezotronics Inc., Buffalo, N.Y.) are used to measure the pressure wave at the end of the expansion chamber and near the animal restraint holders. The shock tube produces a pressure wave that closely mimics the Friedlander wave typically generated in an uncomplicated (i.e., no Mach Stem or other reflective waves) open-field blast environment. Animals are placed into a specially build rodent restrainer at the end of the expansion chamber, or can be placed approximately 5-12 inches into the bore of the expansion chamber depending on experimental requirements.

To evaluate the neuro-behavioral effects of mild-moderate blast injury, three groups of rats were assessed: sham treatment (n=4), primary and tertiary injuries achieved through minimal restraints (BPO+TI; n=2), and primary injury using a full restraint (BPO; n=3). Assessments included a modified Functional Observational Battery (FOB) and a variant of the Morris Water Maze (MWM). Specifically, all animals were trained on the learning set version of the MWM in which the hidden platform is placed in a novel location each day. All animals were trained for four days in the MWM prior to exposure. Animals were anesthetized with tibromoethanol (300 mg/kg, IP) approximately five minutes preceding sham or blast exposure. A Kevlar jacket (0.75 mm thickness) was used to shield the torso and neck of the animal during blast. Animals were placed securely onto a specially built rodent restrainer housed at the exit of the shock tube, perpendicular to the shock front.

Results indicated that average (n=5) measured blast characteristics were consistent with a typical Friedlander wave. Specifically, the average peak overpressure (12.5±2.4 PSI) was consistent with a mild-to-moderate injury range, followed by a rapid decline (peak duration=0.6 ms) pressure and an average impulse of 3.0±0.5 PSI*ms. Results from the MWM task indicated that animals from all three groups reached asymptotic performance after 1-2 trials each day prior to blast exposure. After exposure Sham and BPO animals continued to reach asymptote at 1-2 trials but BPO+TI animals required 4-5 trials, suggesting a difficulty to learn. Moreover, long swim times on the first trial of each day index memory for the previous platform location(s). Prior to blast, all groups had similar persistence. After blast, BPO+TI showed low place persistence, suggesting post-traumatic anterograde and retrograde amnesia for spatial information. Gross necropsy suggested that the only visible abnormalities were noted in the BPO+TI animals. Gross necropsy indicated no signs of neck or internal organ trauma. Finally, average body weight declined in BPO+TI treated animals relative to Sham and BPO groups. In summary, the roles of primary versus primary and tertiary (i.e., traditional acceleration and deceleration) blast-related injuries on neurobehavioral functioning remain actively debated. The current pilot study was designed to both demonstrate basic core competencies as well as examine this critical issue in military mild traumatic brain injury (mTBI).

Additional pilot testing was conducted with seven male C57BL/6J mice (average body weight=29.43+/−4.08 g). Animals were randomly assigned to either SAI (N=5) or sham blast (N=2) conditions. All animals were anesthetized with tibromoethanol (300 mg/kg, IP), and placed in a custom made Kevlar jacket and restraint device for approximately seven minutes. Animals in the SAI condition were placed approximately five inches inside the shock tube bore oriented perpendicular to the shock tube such that the right hemisphere was directly exposed to blast. Sensors were mounted either directly on the bore or the restraint device, side-on to the direction of the shock front to measure static pressure. Summary statistics for the restraint device sensor (FIG. 3A) includes the average maximum peak and duration from the positive and negative phases of the blast exposure, as well as the impulse of the positive phase. Summary statistics for the bore sensor include only the positive phase data (FIG. 3C). Results indicated that the measured shock profiles from the restraint sensor (n=5) were generally consistent with an ideal Friedlander wave function (FIG. 3B) and with previously published results (Goldstein et al., 2012, Sci Transl Med 4:134ra60; Goldstein et al., 2012, Sci Transl Med 4:157lr5). The traces obtained from the bore sensor indicated the true reflective pressure experienced by the animal based on sensor positioning (FIG. 3D). Although the sample size was small, the error bars (standard error of the mean) and individually presented traces (n=5; Panels B and D) demonstrate the high reliability of the exposures across the entire pilot cohort.

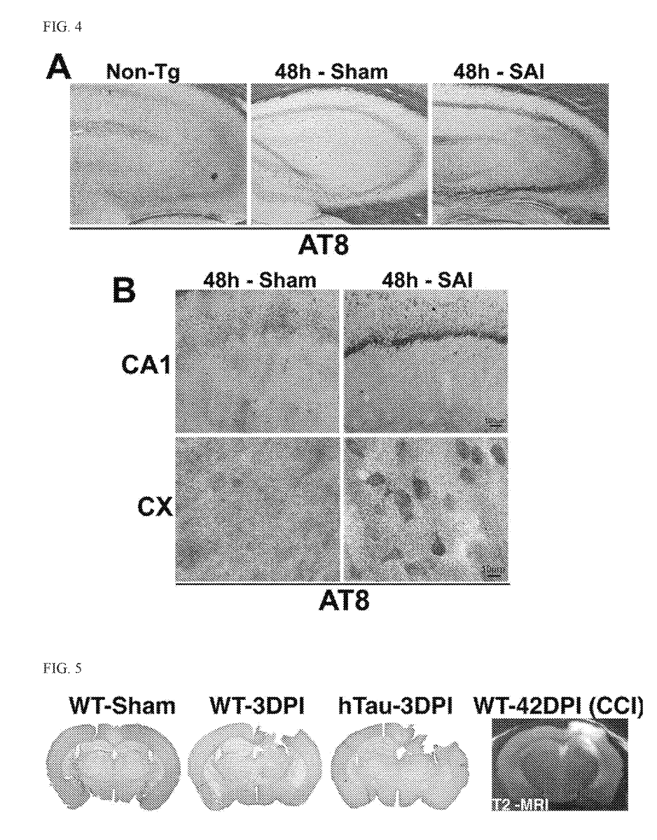

All animals recovered from the effects of anesthesia and airblast/sham exposure in approximately 90 minutes (time to mobility). Animals were euthanized with 0.30 mL of pentobarbital sodium and phenytoin sodium solution (EUTHASOL, Virbac Corp., Fort Worth, Tex.) approximately 48 hours post-exposure. Gross necropsy for all of the animals (cranial, thoracic, and abdominal) was unremarkable, indicating no overt pathology from blast exposure. Average brain weight from the entire cohort was 0.432 g. Free-floating sections from all the mice 48 hours post-injury were processed for immunohistochemistry to detect the presence of hyperphosphorylated MAPT (pS199/pS202 using AT8 monoclonal antibody). Compared to Sham animals, mice subjected to SAI displayed robust AT8+ neurons in the majority of the hippocampus (FIG. 4A). Notably, the CA1 and CA3 neurons of the SAI mice expressed robust AT8 immunoreactivity compared to Sham animals, which displayed very low basal AT8 expression, comparable to uninjured non-transgenic controls (Non-Tg) mice (FIGS. 4A and 4B). AT8+ neurons were also detected in the layers III/IV of the temporal cortex (FIG. 4B). These results suggest that our shock-wave tube device can induce consistent and reliable SAI as well as MAPT hyperphosphorylation similar to those reported by other groups (Goldstein et al., 2012, Sci Transl Med 4:134ra60; Goldstein et al., 2012, Sci Transl Med 4:157lr5).

### Example 2—Lateral Fluid Percussion Injury (FPI)

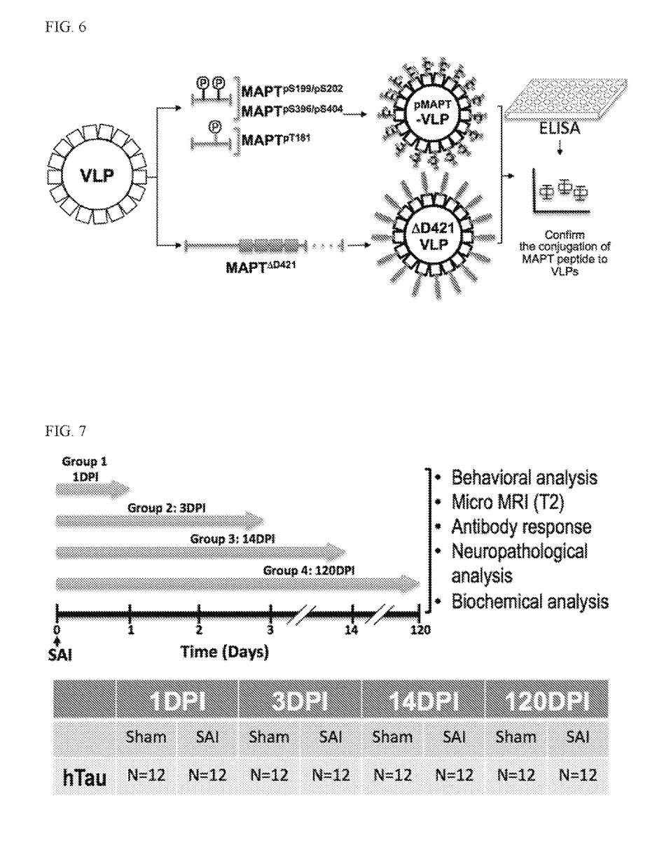

Age-matched and sex-matched 2-month-old nontransgenic C57BL/6J mice (WT) and hTau mice were subjected to moderate to severe lateral fluid percussion injury (FPI) or sham injury (FIG. 5). Photomicrographs of representative brain sections at 3 DPI show a visible injury cavity only in the ipsilateral cortex and part of the hippocampus sparing most of the subcortical regions following fluid percussion injury (FIG. 5).

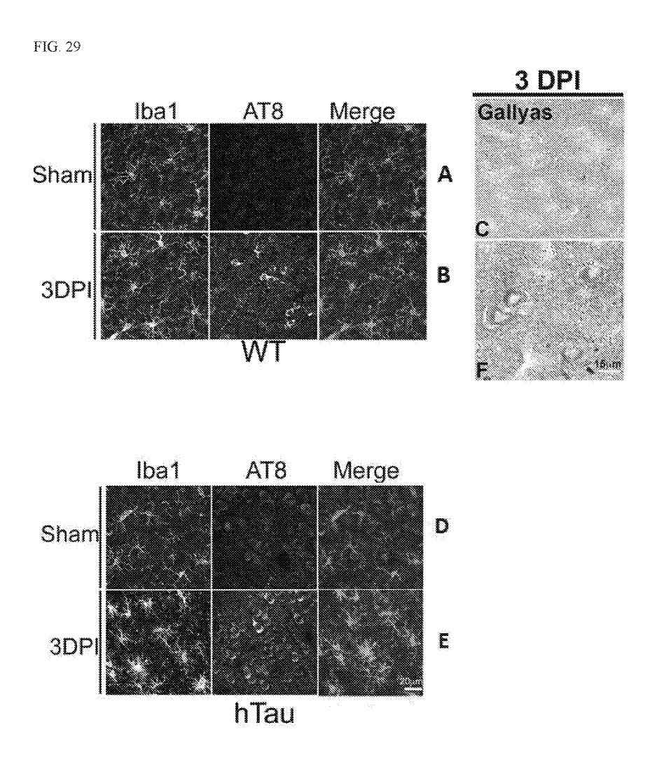

In a separate study, the injury cavity caused by closed contusion injury can be easily visualized by T2-weighted MRI in WT mice at 42 DPI (FIG. 5 right panel). Brain sections from injured WT and hTau mice at 3 DPI stained for microglia (Iba1) and MAPT phosphorylated at S202 (AT8) showed an increase in AT8⁺ dystrophic neurites, neuropil threads and neurons at the site of injury in conjunction with activated Iba1⁺ microglia with shortened processes in both hTau and WT mice (FIG. 29B, E) when compared to sham injured controls (FIG. 29A, D). In addition, numerous Gallyas-positive neurons were identified in the temporal cortex of injured hTau mice (FIG. 29F) but not in brain injured WT mice (FIG. 29C) or sham controls.

### Example 3

### Single Air-Blast Injury (SAI)

Two-month-old hTau mice (in C57BL/6J background; mixed gender) are subjected to mild-to-moderate singe air-blast (SAI) or sham injury. Mild-to-moderate (11 to 15 PSI) SAI (mSAI) mimics pathological characteristics seen in injured veterans in response to one or more blast-related Traumatic Brain Injury. A sample size of n=12 per group (sham, SAI) at each time point (1, 3, 14 and 120 DPI) (total n=96) provides statistical power of 80% and allows an 10% mortality rate. Accordingly, n=12 mice/treatment are used (FIG. 7). Standard procedures described in Example 1, above, and by Goldstein et al. are used to administer SAI or sham injury to mice (Goldstein et al., 2012, Sci Transl Med 4:134ra60; Goldstein et al., 2012, Sci Transl Med 4:157lr5).

After the SAI, the mice are returned to their cage for recovery, and then the mice are subjected to T2 weighted micro-MRI analysis (see below) and diffusion tensor imaging.

Mice in Group 3 and Group 4 (FIG. 7) are subjected to behavioral analysis (see below) at 14 DPI and 120 DPI.

All mice are sacrificed at 1 DPI (Group 1), 3 DPI (Group 2), 14 DPI (Group 3), or 120 DPI (Group 4) via transcardial perfusion (with phosphate buffer), brains from half the mice per group/genotype/treatment (n=6) are immerse-fixed in 4% paraformaldehyde for neuropathological analysis and the brains from the remaining half (n=6) are micro-dissected into cortex (CX), hippocampus (HP) and rest of the brain (ROB) (from both ipsilateral and contralateral sides) and processed for biochemical analysis.

### MRI Analysis

MRI analysis is performed to detect the extent of the brain damage at different time points following SAI on a dedicated MR scanner (4.7T, Bruker Corp., Billerica, Mass.) equipped with single tuned surface coil for mouse brain (RAPID Biomedical GmbH, Rimpar, Germany). The mice are anesthetized using isoflurane gas (induction dosage 2-3%; maintenance dosage 1.5-2%) and a mixture of O₂:N₂O gases in the ratio 2:1, delivered during the measurements. Real-time monitoring of physiological parameters (heart rate and respiratory rate) is performed during the entire duration of the study. A tri-pilot scan using gradient echo sequence acquires initial localizer images. T2-weighted MRI is performed with a fast spin-echo sequence (RARE), TR/TE=5000/56 ms, FOV=4 cm×4 cm, Slice thickness=1 mm, Inter-slice distance=1 mm, number of slices=12, matrix=256×256, number of average=6. Diffusion tensor imaging is performed with an echo-planar imaging sequence, TR/TE=3800/40 ms, FOV=4 cm×4 cm, Slice thickness=1 mm, number of average=3 with 30 unique diffusion directions and the b=0 experiment repeated 3 times per average.

### Behavioral Analysis

### 1. Spatial Alternation in the Y-Maze:

Spontaneous alternation in the Y-maze is a rapid and accurate measure of working spatial memory (Hughes, R. N., 2004, Neurosci Biobehav Rev 28:497-505) in mouse models of Alzheimer's disease. Each animal is placed in the center of the Y-maze and allowed free exploration for five minutes. The total number of arm choices and number of spontaneous alternations, where the previous two arm choices differed from the third, is calculated from the videotaped session.

### 2. Novel Object Recognition Test

After the Y-maze test, Novel Object Recognition test, a measure of recognition memory, which is significantly impaired in hTau mice at 12 months of age, is completed over three days as previously described (Oliveira et al., 2010, Learn Mem 17:155-160) with minor modifications. Briefly, on day one, animals are placed in an open arena (60 cm×50 cm×40 cm) for a 10-minute habituation. On day two, animals are placed in the same open arena and exposed to two similar objects for 10 minutes. On day three, animals are placed in the same open arena and exposed to one familiar object (exactly the same as the objects from day two) and one novel object (distinct in shape and texture from the familiar object) for 10 minutes. A video tracking system (ETHOVISION, Noldus Information Technology, Leesburg, Va.) is used to calculate the percentage of time spent on object exploration.

### 3. Morris Water Maze (MWM)

Mice are tested in the hidden platform task of the Morris Water Maze as described (Morris, R., 1984, J Neurosci Methods 11:47-60). The mice are trained to find a hidden platform for five days with a 20-minute inter-trial interval. After the mice have been trained to find the platform, the platform is removed and the mice perform a probe trial, where they are given one minute to investigate the water maze. A video tracking system (ETHOVISION, Noldus Information Technology, Leesburg, Va.) is used to monitor the time spent in each quadrant and measure the spatial bias of the animal's search pattern to reflect spatial learning.

### Neuropathological Analysis

Fixed brain tissue is cryoprotected (a 20% glycerol solution) and frozen before preparing coronal free-floating sections for immunohistochemistry. For studies of MAPT pathology, sections are processed to detect early (AT8, AT180, and MC1) and late (PHF1, Alz50) disease-specific MAPT epitopes, truncated (ΔD421), and MAPT aggregates (detected using Gallyas silver staining; Bhaskar et al., 2010, Neuron 68:19-31). Additional sections are used to identify neurodegeneration (annexin V, caspase 3, and Tunnel) via double immunofluorescence with a neuron-specific antigen NeuN. Neuroinflammation is detected via specific antibodies (microglia/macrophage by Iba1, CD45, CD68, F4/80; astrocytes by GFAP). Images will be captured in the ZEISS Meta inverted fluorescent microscope, and the quantifications will be performed utilizing Stereo Investigator® (MBF Bioscience, Williston, Vt.).

### Biochemical Analysis

Antibody responses in the serum are quantified as previously described (Chackerian et al., 2006, Vaccine 24:6321-6331). For MAPT biochemistry, proteins from the frozen brains are extracted in Tissue-Protein Extraction Reagent (T-PER, Pierce, Thermo Fisher Scientific, Inc., Waltham, Mass.) with protease and phosphatase inhibitor cocktails and Western blot analysis performed. Specific antibodies to phosphorylated MAPT pS199/S202 (AT8), pT231 (AT180), pS396/pS404 (PHF1*, Dickson et al., 1987, Acta Neuropathol 73:254-258), conformational epitopes (Alz50*, Carmel et al., 1996, J Boil Chem 271:32789-32795; MC1*, Jicha et al., 1997, J Neurosci Res 48:128-132), truncated MAPT (ΔD421, Millipore MAB5430), and total MAPT (Taus and Tau12—a human MAPT specific antibody) are used to detect respective phosphorylated MAPT. Appropriate secondary antibodies are used for immunodetection on Western blots (Bhaskar et al., 2010, Neuron 68:19-31).

### Example 4

Inoculation of hTau Mice with MAPT-VLPs

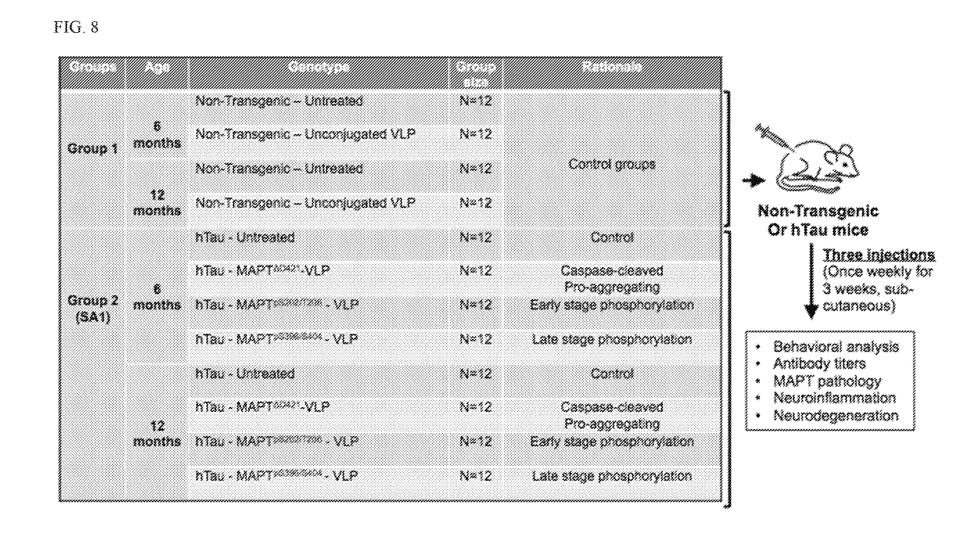

To assess the effectiveness of MAPT-VLPs in reducing the levels of truncated-MAPT, hyperphosphorylated-MAPT, and conformationally disordered-MAPT in hTau mice following SAI, 2-month old hTau mice are subjected to SAI as described in Example 3, then aged until they display robust MAPT pathology (as described in Example 3). Three weeks prior to peak MAPT pathology following SAI, injured hTau mice are inoculated intramuscularly with respective MAPT-VLPs once weekly for three weeks (FIG. 8).

After last serum collection, the mice are subjected to behavioral analysis, micro-MRI analysis (as described in Example 3) and sacrificed via transcardial perfusion with ice-cold phosphate buffer. The brains from half the mice in each group are microdissected into CX, HP, and ROB, weighed and frozen in liquid nitrogen for later biochemical analysis as described in Example 3. The brains from the other half of the mice in each group are immersion fixed in 4% paraformaldehyde for later neuropathological analysis as described in Example 3.

### Example 5

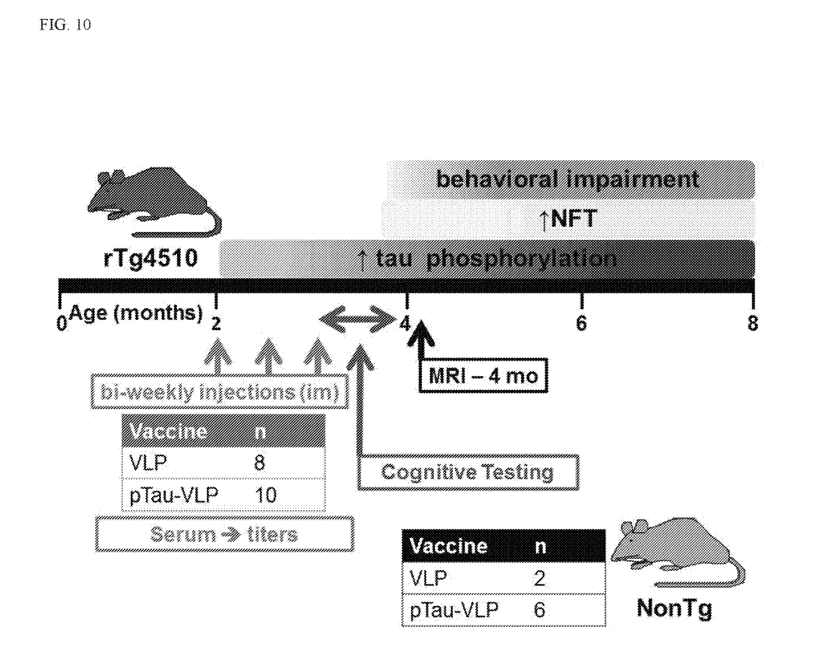

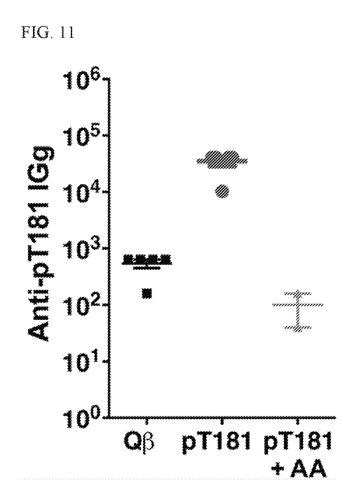

The immunization protocol shown in FIG. 10, including 3 biweekly intramuscular (im) injections, was carried out on 8 Non-Tg mice, 2 with VLP and 6 with pTau-VLP, and on 18 rTg4510 mice, 8 with VLPs alone and 10 with pTau-VLP. The serum was checked for the presence of antibody after the second injection. After treatment, the animals were given a battery of cognitive tests and were sacrificed for biochemical analysis.

### Qβ-MAPT^{pT181} Vaccination Study

The anti-MAPT^{pT181} IgG response generated in response to vaccinating with MAPT^{pT181} in the absence of the Qβ VLP platform but with a standard alum adjuvant (AA) was compared to anti-MAPT^{pT181}IgG response generated in response to vaccinating with Qβ-MAPT^{pT181} or Qβ alone.

To assess the brain penetrance of MAPT^{pT181} IgG, ELISA was performed on serum and brain (cortex) lysates of mice immunized with Qβ-MAPT^{pT181} or Qβ alone (FIG. 12 A, B). Notably, the level of pT181-reactive IgG was significantly higher in the brain lysates of mice immunized with Qβ-MAPTpT181 than mice immunized with Qβ alone (FIG. 12B). Furthermore, the presence of pT181 IgG in the brain was confirmed by performing reverse immunohistochemistry. The brain sections were first incubated with biotinylated-T181 peptide and then incubated with streptavidin-conjugated to horse-radish peroxidase. Following developing the color reaction with a substrate, the presence of brown precipitation within several cells in the hippocampal dentate gyrus region was observed (FIG. 12C).

To assess the effect of vaccination on cognitive function, the novel object recognition test was performed as described in Example 3. Briefly, wild type mice typically spend about 80% of their time with the novel object. In contrast, untreated rTg4510 mice typically spend relatively equal amounts of time with both the novel object and the familiar object. Vaccination with Qβ-MAPT^{pT181} rescues some cognitive function; vaccinated rTg4510 mice show a statistically significant increased preference to the novel object (compared to a familiar object) (FIG. 13A).

Proteins from frozen brains of rTg4510 mice vaccinated with Qβ-MAPT^{pT181} or Qβ were extracted using Tissue-Protein Extraction Reagent (T-PER, Pierce, Thermo Fisher Scientific, Inc., Waltham, Mass.) with protease and phosphatase inhibitor cocktails, and Western blot analysis was performed (FIG. 14A). Antibodies specific to phosphorylated MAPT pS199/S202 (AT8) showed that rTg4510 mice vaccinated with Qβ-MAPT^{pT181} displayed significantly reduced levels of AT8+ MAPT compared to Qβ-injected rTg4510 mice, as measured by Western Blot (FIG. 14A, B) and immunohistochemistry (FIG. 14C).

MRI analysis (4.7T) of rTg4510 mice vaccinated with Qβ-MAPT^{pT181} or Qβ demonstrated that, with age, Qβ-MAPT^{pT181}mice show a trend towards reduced brain atrophy (T2 weighted images, FIG. 15, FIG. 16).

4 month old rTg4510 mice treated with Qβ-alone showed statistically significant cortical atrophy compared to 4 month old non-transgenic mice. This atrophy is rescued in 4 month old rTg4510 mice treated with Qβ-MAPT^{pT181}(FIG. 16, bottom panel; n=5 in each group). Two group comparative analysis via unpaired student t-test showed statistically significant rescue in cortical atrophy in 4 month old rTg4510 mice treated with Qβ-MAPT^{pT181} compared to Qβ treated control mice (FIG. 16, bottom panel). A trend towards reduced positive correlation between apparent diffusion coefficient (ADC) and fractional anisotropy (FA) indicates protection from white matter damage (FIG. 28).

Similar to quantification of Gallyas silver positive tangles in the cortex (FIGS. 2 B, D, and E), the presence of Gallyas silver positive tangles in the hippocampus was examined and quantified (FIG. 17). A statistically significant reduction in the number of silver positive tangles in the Qβ-MAPTpT181 treated mice was observed compared to control group (FIG. 17).

A Sarkosyl insoluble assay (performed as described in Bhaskar et al., Neuron 2010, 68(1):19-31; Maphis et al., Brain 2015, 138(Pt 6):1738-55; Mocanu et al., J Neurosci. 2008, 28(3):737-748) was performed to determine if Qβ-MAPT^{pT181} vaccination results in reduced insoluble/aggregated tau/tangles. Significantly reduced Sarkosyl insoluble tangles were observed in the Qβ-MAPT^{pT181} mice compared to Qβ-treated control mice (FIG. 18).

Immunofluorescence analysis to detect NeuN (a marker of neurons) and TUNEL (a marker for apoptotic cells) was performed to determine if Qβ-MAPT^{pT181} vaccination reduces neurodegeneration/neuronal loss. Results suggest that mice vaccinated with Qβ-MAPT^{pT181} show reduced numbers of TUNEL & NeuN double positive cells in the hippocampus compared to rTg4510 mice vaccinated with Qβ (FIG. 19).

To determine if vaccination with Qβ-MAPT^{pT181} is likely to cause hemorrhage/edema, brain sections from vaccinated mice were stained with Haematoxylin and Eosin (H & E) and compared to brain sections from spontaneously-hypertensive stroke prone rats (SHR-SP rats), which display significant level of brain hemorrhage. Neither Qβ-MAPT^{pT181} nor control Qβ vaccinated mice showed any evidence of hemorrhage in the brain (FIG. 20).

Qβ-MAPT^{pS396/s404} and Qβ-MAPT^{p199/s202} Vaccination Studies

Qβ-MAPT^{pS396/S404} and Qβ-MAPT^{pS199/S202} were evaluated as VLP-based vaccine candidates in separate cohorts of 2 month old rTg4510 mice.

2-month-old rTg4510 mice vaccinated with Qβ-MAPTP^{S396/S404} showed modest, but not statistically significant, reductions in tau phosphorylated at AT8 (S199/S202) and AT180 (T231) sites (FIG. 21 A, B). Qβ-MAPTP^{S396/S404} immunized mice showed improved recognition memory in the novel object recognition test (i.e., can spent significantly more time with novel object compared to familiar object), performed as described in Example 3 (FIG. 13B).

2-month old rTg4510 mice vaccinated with Qβ-MAPTP^{S199/S202} also spent significantly more time with novel object compared to familiar object on a test day (day 3) of the novel object recognition test, performed as described in Example 3 (FIG. 13C).

### Example 6

Construction of the hTauCamKIIαCreMyD88^{f/f} Mouse

B6.Cg-Tg(Camk2α-cre)T29-1Stl/J mice (The Jackson Laboratory, Bar Harbor, Me.), hereafter referred to as CamK2αCre), hTau mice (Andorfer et al., 2003, J Neurochem 86:582-590), and mice expressing foxed allele of MyD88 (Yu et al., 2014, J Exp Med 211:887-907) were crossed as depicted in FIG. 26 to produce hTauCamK2αCreMyD88^{f/f} mice.

### Example 7

hTauCamK2αCreMyD88^{f/f} mice and hTau mice were placed in an open arena (60 cm×50 cm×40 cm) for 10 minutes of habituation in Day 1. On Day 2, animals were placed in the same open arena and exposed to two similar objects for 10 minutes. On Day 3, animals were placed in the same open arena and exposed to one familiar object (from day two) and one novel object, distinct in shape and texture from the familiar object, for 10 minutes. The time that the animal spent memory exploring each object was recorded.

Results are shown in FIG. 25B.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

### Sequence Listing Free Text

| ID | Name | Sequence# |
|---|---|---|
| SEQ. ID NO. 1 | MAPT^{pT181} | TPPAPKT*PPSSGE |
| SEQ. ID NO. 2 | MAPT^{pS199/pS202} | GDRSGYSS*PGS*PGTPGSR |
| SEQ. ID NO. 3 | MAPT^{pT231} | KVAVVRT*PPKSPSS |
| SEQ. ID NO. 4 | MAPT^{pS396/pS404} | AEIVYKS*PVVSGDTS*PRHLSN |
| SEQ. ID NO. 5 | MAPT^{ΔD421} | MAEPRQEFEV MEDHAGTYGL GDRKDQGGYT MHQDQEGDTD |
| | | AGLKESPLQT PTEDGSEEPG SETSDAKSTP TAEDVTAPLV |
| | | DEGAPGKQAA AQPHTEIPEG TTAEEAGIGD TPSLEDEAAG |
| | | HVTQARMVSK SKDGTGSDDK KAKGADGKTK IATPRGAAPP |
| | | GQKGQANATR IPAKTPPAPK TPPSSGEPPK SGDRSGYSSP |
| | | GSPGTPGSRS RTPSLPTPPT REPKKVAVVR TPPKSPSSAK |
| | | SRLQTAPVPM PDLKNVKSKI GSTENLKHQP GGGKVQIINK |
| | | KLDLSNVQSK CGSKDNIKHV PGGGSVQIVY KPVDLSKVTS |
| | | KCGSLGNIHH KPGGGQVEVK SEKLDFKDRV QSKIGSLDNI |
| | | THVPGGGNKK IETHKLTFRE NAKAKTDHGA EIVYKSPVVS |
| | | GDTSPRHLSN VSSTGSIDMV D |
| SEQ. ID NO. 6 | MAPT^{pT210/pS214} | SRT*PS*LP |
| SEQ. ID NO. 7 | MAPT^{pS262} | IGS*TE |
| SEQ. ID NO. 8 | MAPT^{pT231/pS235} | VRT*PPKS*PS |
| SEQ. ID NO. 9 | MAPT^{pS396/pSS404} | YKS*PVVSGDTS*PR |
| SEQ. ID NO. 10 | MAPT^{pT212/pS214} | GDRSGYSSPGSPGTPGSRSRT* PS*LPTPPTR |
| SEQ. ID NO. 11 | MAPT^{pS199/pS202/pT205/pT212/pS214} | GDRSGYSS*PGS*PGT* PGSRSRT*PS*LPTPPTR |
| SEQ. ID NO. 12 | MAPT^{pS199/pT212/pS214/pT217} | GDRSGYSS*PGSPGTPGSRSRT* PS*LPT*PPTR |
| SEQ. ID NO. 13 | MAPT^{pS202/pT205} | GDRSGYSSPGS*PGT* PGSRSRTPSLPTPPTR |
| SEQ. ID NO. 14 | MAPT^{pT212/pS214} | PGSPGTPGSRSRT*PS* LPTPPTREPKKVAVV |
| SEQ. ID NO. 15 | MAPT^{pS234/pS356} | CGS*LGNIHHKPGGGQV EVKSEKLDFKDRVQSKIGS* LD |
| SEQ. ID NO. 16 | MAPT^{pS262} | IGS*TENLKHQPG |
| SEQ. ID NO. 17 | MAPT^{pT50/T59} | LQT*PTEDGSEEPG SETSDAKST*PT |
| SEQ. ID NO. 18 | MAPT^{pS113} | TPS*LE |
| SEQ. ID NO. 19 | MAPT^{pT153} | IAT*PR |
| SEQ. ID NO. 20 | MAPT^{pT175} | AKT*PP |
| SEQ. ID NO. 21 | MAPTp^{T205/pS208/pS210/pT212/pS214/pT217} | PGT*PGS*RS*RT* PS*LPT*PP |
| SEQ. ID NO. 22 | MAPT^{pT235} | PKS*PS |
| SEQ. ID NO. 23 | MAPT^{pS258/pS262} | VKS*KIGS*TE |
| SEQ. ID NO. 24 | MAPT^{pS289} | VQS*KC |
| SEQ. ID NO. 25 | MAPT^{pS356} | IGS*LD |
| SEQ. ID NO. 26 | MAPT^{pS400/pT403/pS409/pS412/pT414/pS435} | VVS*GDT*SPRHLS*NVS* S*T*GS |
| SEQ. ID NO. 27 | MAPT^{pS422/pT427/pS433/pS435} | VDS*PQLAT*LADEVS* AS*LA |
| SEQ. ID NO. 28 | MAPT^{Ps292} | PGS*P |
| SEQ. ID NO. 29 | MAPT^{pT295} | PGT*PG |
| SEQ. ID NO. 30 | MAPT^{pS199/pS202/pT205} | YSS*PGS*PGT*PG |
| SEQ. ID NO. 31 | MAPT^{pT212/pS214} | PGSRSRT*PS*LP |
| SEQ. ID NO. 32 | MAPT^{pT231/pS235} | VRT*PPKS*PSSA |
| SEQ. ID NO. 33 | MAPT^{pT181/pS184/pS185} | PKT*PPS*S*GEP |

*(PO₃H₂)
# Any amino acid sequence can have a C-terminal GGC appended to facilitate conjugation.

What is claimed is:

1. An immunogen comprising:
an antigen presentation component; and
a microtubule-associated tau protein (MAPT) component linked to at least a portion of the antigen presentation component.

2. The immunogen of claim 1 wherein the MAPT component comprises at least one amino acid residue modified to comprise a PO₃H₂ group.

3. The immunogen of claim 2 wherein the MAPT component comprises the amino acid sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33.

4. The immunogen of any preceding claim wherein the antigen presentation component comprises a virus-like particle (VLP).

5. The immunogen of any preceding claim wherein the VLP comprises bacteriophage Qβ or MS2.

6. The immunogen of any preceding claim wherein the antigen presenting component and the MAPT component are linked covalently.

7. The immunogen of claim 6 wherein the covalent link comprises a succinimidyl-6-[β-maleimidopropionamido]hexanoate (SMPH) linkage.

8. A pharmaceutical composition comprising the immunogen of any preceding claim.

9. The pharmaceutical composition of claim 8 further comprising an adjuvant.

10. A method of treating a subject having or at risk of having a tauopathic condition, the method comprising:
administering to the subject an amount of the immunogen of any one of claims 1-7 effective to ameliorate at least one symptom or clinical sign of the tauopathic condition.

11. The method of claim 10 wherein the tauopathic condition comprises Alzheimer's disease, progressive supranuclear palsy (PSP), corticobasal degeneration (CBD), Pick's disease (PiD), frontotemporal dementia and Parkinsonism linked to chromosome-17 Tau Type (FTDP-17T), argyrophilic grain dementia (AGD), traumatic brain injury (TBI), or chronic traumatic encephalopathy (CTE).

12. The method of claim 10 or claim 11 wherein the symptom or clinical sign of the tauopathic condition comprises neurodegeneration or cognitive impairment.

13. The method of any one of claims 10-12 further comprising at least one anti-inflammatory strategy.

14. The method of claim 13 wherein the anti-inflammatory strategy comprises:
enrichment of IgG4 immunoglobulins;
removing RNA from the VLP component; or
enrichment of regulatory B cells that express IL-10.

15. The method of any one of claims 10-14 wherein the treatment is prophylactic.

16. The method of any one of claims 10-14 wherein the treatment is therapeutic.

17. A polynucleotide that encodes the immunogen of any one of claims 1-7.

18. A cell comprising the polynucleotide of claim 17.

19. A transgenic mouse line comprising:
brain cells that comprise:
a polynucleotide that encodes human microtubule-associated protein tau (MAPT); and
a deletion of at least a portion of endogenous mouse MAPT; and
a forebrain neuron-specific deletion of a polynucleotide that encodes Myeloid Differentiation Primary Response Gene 88 (MyD88).

20. The transgenic mouse of claim 19 wherein brain neurons exhibit reduced response to IL-1β.


SEQUENCE LISTING (33 sequences)

SEQ ID NO: 1 - protein, 16 aa, artificial
  synthetic peptide
  MISC_FEATURE (7)..(7): PO3H2
  MISC_FEATURE (14)..(16): This region may include no residues or GGC.
TPPAPKTPPSSGEXXX

SEQ ID NO: 2 - protein, 21 aa, artificial
  synthetic peptide
  MISC_FEATURE (8)..(8): PO3H2
  MISC_FEATURE (11)..(11): PO3H2
  MISC_FEATURE (19)..(21): This region may include no residues or GGC.
GDRSGYSSPGSPGTPGSRXXX

SEQ ID NO: 3 - protein, 17 aa, artificial
  synthetic peptide
  MISC_FEATURE (7)..(7): PO3H2
  MISC_FEATURE (15)..(17): This region may include no residues or GGC.
KVAVVRTPPKSPSSXXX

SEQ ID NO: 4 - protein, 24 aa, artificial
  synthetic peptide
  MISC_FEATURE (7)..(7): PO3H2
  MISC_FEATURE (15)..(15): PO3H2
  MISC_FEATURE (22)..(24): This region may include no residues or GGC.
AEIVYKSPVVSGDTSPRHLSNXXX

SEQ ID NO: 5 - protein, 424 aa, artificial
  synthetic peptide
  MISC_FEATURE (422)..(424): This region may include no residues or GGC.
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDXXX

SEQ ID NO: 6 - protein, 10 aa, artificial
  synthetic peptide
  MISC_FEATURE (3)..(3): PO3H2
  MISC_FEATURE (5)..(5): PO3H2
  MISC_FEATURE (8)..(10): This region may include no residues or GGC.
SRTPSLPXXX

SEQ ID NO: 7 - protein, 8 aa, artificial
  synthetic peptide
  MISC_FEATURE (3)..(3): PO3H2
  MISC_FEATURE (6)..(8): This region may include no residues or GGC.
IGSTEXXX

SEQ ID NO: 8 - protein, 12 aa, artificial
  synthetic peptide
  MISC_FEATURE (3)..(3): PO3H2
  MISC_FEATURE (7)..(7): PO3H2
  MISC_FEATURE (10)..(12): This region may include no residues or GGC.
VRTPPKSPSXXX

SEQ ID NO: 9 - protein, 16 aa, artificial
  synthetic peptide
  MISC_FEATURE (3)..(3): PO3H2
  MISC_FEATURE (11)..(11): PO3H2
  MISC_FEATURE (14)..(16): This region may include no residues or GGC.
YKSPVVSGDTSPRXXX

SEQ ID NO: 10 - protein, 33 aa, artificial
  synthetic peptide
  MISC_FEATURE (21)..(21): PO3H2
  MISC_FEATURE (23)..(23): PO3H2
  MISC_FEATURE (31)..(33): This region may include no residues or GGC.
GDRSGYSSPGSPGTPGSRSRTPSLPTPPTRXXX

SEQ ID NO: 11 - protein, 33 aa, artificial; identical to SEQ ID NO: 10
  synthetic peptide
  MISC_FEATURE (8)..(8): PO3H2
  MISC_FEATURE (11)..(11): PO3H2
  MISC_FEATURE (14)..(14): PO3H2
  MISC_FEATURE (21)..(21): PO3H2
  MISC_FEATURE (23)..(23): PO3H2
  MISC_FEATURE (31)..(33): This region may include no residues or GGC.

SEQ ID NO: 12 - protein, 33 aa, artificial; identical to SEQ ID NO: 10
  synthetic peptide
  MISC_FEATURE (8)..(8): PO3H2
  MISC_FEATURE (21)..(21): PO3H2
  MISC_FEATURE (23)..(23): PO3H2
  MISC_FEATURE (26)..(26): PO3H2
  MISC_FEATURE (31)..(33): This region may include no residues or GGC.

SEQ ID NO: 13 - protein, 33 aa, artificial; identical to SEQ ID NO: 10
  synthetic peptide
  MISC_FEATURE (11)..(11): PO3H2
  MISC_FEATURE (14)..(14): PO3H2
  MISC_FEATURE (31)..(33): This region may include no residues or GGC.

SEQ ID NO: 14 - protein, 33 aa, artificial
  synthetic peptide
  MISC_FEATURE (13)..(13): PO3H2
  MISC_FEATURE (15)..(15): PO3H2
  MISC_FEATURE (31)..(33): This region may include no residues or GGC.
PGSPGTPGSRSRTPSLPTPPTREPKKVAVVXXX

SEQ ID NO: 15 - protein, 40 aa, artificial
  synthetic peptide
  MISC_FEATURE (3)..(3): PO3H2
  MISC_FEATURE (35)..(35): PO3H2
  MISC_FEATURE (38)..(40): This region may include no residues or GGC.
CGSLGNIHHKPGGGQVEVKSEKLDFKDRVQSKIGSLDXXX

SEQ ID NO: 16 - protein, 15 aa, artificial
  synthetic peptide
  MISC_FEATURE (3)..(3): PO3H2
  MISC_FEATURE (13)..(15): This region may include no residues or GGC.
IGSTENLKHQPGXXX

SEQ ID NO: 17 - protein, 27 aa, artificial
  synthetic peptide
  MISC_FEATURE (3)..(3): PO3H2
  MISC_FEATURE (22)..(22): PO3H2
  MISC_FEATURE (25)..(27): This region may include no residues or GGC.
LQTPTEDGSEEPGSETSDAKSTPTXXX

SEQ ID NO: 18 - protein, 8 aa, artificial
  synthetic peptide
  MISC_FEATURE (3)..(3): PO3H2
  MISC_FEATURE (6)..(8): This region may include no residues or GGC.
TPSLEXXX

SEQ ID NO: 19 - protein, 8 aa, artificial
  synthetic peptide
  MISC_FEATURE (3)..(3): PO3H2
  MISC_FEATURE (6)..(8): This region may include no residues or GGC.
IATPRXXX

SEQ ID NO: 20 - protein, 8 aa, artificial
  synthetic peptide
  MISC_FEATURE (3)..(3): PO3H2
  MISC_FEATURE (6)..(8): This region may include no residues or GGC.
AKTPPXXX

SEQ ID NO: 21 - protein, 20 aa, artificial
  synthetic peptide
  MISC_FEATURE (3)..(3): PO3H2
  MISC_FEATURE (6)..(6): PO3H2
  MISC_FEATURE (8)..(8): PO3H2
  MISC_FEATURE (10)..(10): PO3H2
  MISC_FEATURE (12)..(12): PO3H2
  MISC_FEATURE (15)..(15): PO3H2
  MISC_FEATURE (18)..(20): This region may include no residues or GGC.
PGTPGSRSRTPSLPTPPXXX

SEQ ID NO: 22 - protein, 8 aa, artificial
  synthetic peptide
  MISC_FEATURE (3)..(3): PO3H2
  MISC_FEATURE (6)..(8): This region may include no residues or GGC.
PKSPSXXX

SEQ ID NO: 23 - protein, 12 aa, artificial
  synthetic peptide
  MISC_FEATURE (3)..(3): PO3H2
  MISC_FEATURE (7)..(7): PO3H2
  MISC_FEATURE (10)..(12): This region may include no residues or GGC.
VKSKIGSTEXXX

SEQ ID NO: 24 - protein, 8 aa, artificial
  synthetic peptide
  MISC_FEATURE (3)..(3): PO3H2
  MISC_FEATURE (6)..(8): This region may include no residues or GGC.
VQSKCXXX

SEQ ID NO: 25 - protein, 8 aa, artificial
  synthetic peptide
  MISC_FEATURE (3)..(3): PO3H2
  MISC_FEATURE (6)..(8): This region may include no residues or GGC.
IGSLDXXX

SEQ ID NO: 26 - protein, 22 aa, artificial
  synthetic peptide
  MISC_FEATURE (3)..(3): PO3H2
  MISC_FEATURE (6)..(6): PO3H2
  MISC_FEATURE (12)..(12): PO3H2
  MISC_FEATURE (15)..(15): PO3H2
  MISC_FEATURE (16)..(16): PO3H2
  MISC_FEATURE (17)..(17): PO3H2
  MISC_FEATURE (20)..(22): This region may include no residues or GGC.
VVSGDTSPRHLSNVSSTGSXXX

SEQ ID NO: 27 - protein, 21 aa, artificial
  synthetic peptide
  MISC_FEATURE (3)..(3): PO3H2
  MISC_FEATURE (8)..(8): PO3H2
  MISC_FEATURE (14)..(14): PO3H2
  MISC_FEATURE (16)..(16): PO3H2
  MISC_FEATURE (19)..(21): This region may include no residues or GGC.
VDSPQLATLADEVSASLAXXX

SEQ ID NO: 28 - protein, 7 aa, artificial
  synthetic peptide
  MISC_FEATURE (3)..(3): PO3H2
  MISC_FEATURE (5)..(7): This region may include no residues or GGC.
PGSPXXX

SEQ ID NO: 29 - protein, 8 aa, artificial
  synthetic peptide
  MISC_FEATURE (3)..(3): PO3H2
  MISC_FEATURE (6)..(8): This region may include no residues or GGC.
PGTPGXXX

SEQ ID NO: 30 - protein, 14 aa, artificial
  synthetic peptide
  MISC_FEATURE (3)..(3): PO3H2
  MISC_FEATURE (6)..(6): PO3H2
  MISC_FEATURE (9)..(9): PO3H2
  MISC_FEATURE (12)..(14): This region may include no residues or GGC.
YSSPGSPGTPGXXX

SEQ ID NO: 31 - protein, 14 aa, artificial
  synthetic peptide
  MISC_FEATURE (7)..(7): PO3H2
  MISC_FEATURE (9)..(9): PO3H2
  MISC_FEATURE (12)..(14): This region may include no residues or GGC.
PGSRSRTPSLPXXX

SEQ ID NO: 32 - protein, 14 aa, artificial
  synthetic peptide
  MISC_FEATURE (3)..(3): PO3H2
  MISC_FEATURE (7)..(7): PO3H2
  MISC_FEATURE (12)..(14): This region may include no residues or GGC.
VRTPPKSPSSAXXX

SEQ ID NO: 33 - protein, 13 aa, artificial
  synthetic peptide
  MISC_FEATURE (3)..(3): PO3H2
  MISC_FEATURE (6)..(6): PO3H2
  MISC_FEATURE (7)..(7): PO3H2
  MISC_FEATURE (11)..(13): This region may include no residues or GGC.
PKTPPSSGEPXXX